(19) United States
(12) Patent Application Publication
(10) Pub. No.: US 2025/0205404 A1
(43) Pub. Date: Jun. 26, 2025

(54) SYSTEM AND METHOD FOR AUTOMATED ASSESSMENT OF PERITONEAL MEMBRANE FUNCTION, RESIDUAL CAVITY VOLUME, AND PERITONEAL ADEQUACY

(71) Applicants: Fresenius Medical Care Holdings, Inc., Waltham, MA (US); Fresenius Medical Care Deutschland GmbH, Bad Homburg (DE)

(72) Inventors: Paul Chamney, Tring (GB); David Yuds, Hudson, NH (US); Eric Bergman, Newton, MA (US); Kerissa Adams, Norman, OK (US); Jun Yi, New York, NY (US)

(21) Appl. No.: 18/985,834

(22) Filed: Dec. 18, 2024

Related U.S. Application Data
(60) Provisional application No. 63/612,750, filed on Dec. 20, 2023.

(51) Int. Cl. A61M 1/28; G16H 10/60; G16H 20/40; G16H 50/20

(52) U.S. Cl. CPC A61M 1/282; G16H 10/60; G16H 20/40; G16H 50/20; A61M 2205/3379; A61M 2230/20; A61M 2230/201

(57) ABSTRACT

This disclosure teaches methods and systems for determining membrane transport parameters of a peritoneal dialysis (PD) patient, and updating a (PD) prescription of the patient based on one or more of the membrane transport parameters. The methods include withdrawing a plurality of samples of PD effluent from the patient's peritoneal cavity periodically during one or more dwell phases of one or more PD treatment cycles. The method further includes obtaining sensor measurements of the samples of PD effluent, determining one or more peritoneal membrane transport parameters based on the sensor measurements, and updating the patient's PD prescription, to be used for a later peritoneal dialysis treatment, based on the one or more peritoneal membrane transport parameters.

### CROSS-REFERENCE TO RELATED APPLICATIONS

This patent application claims the benefit of U.S. Provisional Patent Application No. 63/612,750, filed Dec. 20, 2023, which is incorporated by reference herein in its entirety.

### BACKGROUND

When renal function becomes impaired beyond a point where homeostasis can no longer be maintained, renal replacement therapy (RRT) is an essential life sustaining intervention for many patients. One form of RRT, suited for treatment of patients in the home setting is peritoneal dialysis (PD). In PD, the membranous lining of the patient's peritoneum acts as a natural semi-permeable membrane that allows the passage of water and solutes. At the start of a PD cycle, dialysis fluid (e.g., fresh PD fluid) is instilled in the peritoneal cavity, establishing an osmotic gradient between the dialysis fluid and the surrounding body tissues. This causes waste products, excess sodium and water to be drawn from body tissues into the peritoneal cavity. After a period of several hours, the osmotic gradient dissipates and the transfer of waste products and excess sodium in particular ceases. At this point, the spent dialysis fluid (e.g., PD effluent) is drained from the cavity denoting the end of the PD cycle. Usually, several cycles are required each day. The amount of solute, water and sodium removed on a weekly basis by PD, is determined by the ‘dose’ variables of a PD prescription, the transport status of the peritoneal membrane, and the efficiency with which the peritoneal cavity can be drained of spent dialysis fluid.

Although the PD process may be understood in qualitative terms, it is generally difficult to control the process accurately in the individual patient, in view of the methods available in today's standard of care. Consequently, when complications of PD such as peritonitis and catheter malfunction are mitigated, technique survival is ultimately limited by the onset of cardiovascular events, and/or failure of the peritoneal membrane upon which the PD process is completely dependent. Furthermore, as objective methods to control the PD process are lacking, then regarding improvement to quality of life, there is very little scope to offer the patient more flexible treatment schedules.

Incremental PD serves to deliver sufficient dialysis such that when combined with a patient's residual kidney function, overall adequacy outcome metrics are met. Thus, the process of preparing a PD prescription in the individual patient, requires prior identification of adequacy targets for water, solute and sodium removal. To establish the dose variables of a PD prescription cycle (e.g., fill volume, glucose composition, and dwell duration) that deliver the adequacy targets is a major challenge. As adequacy metrics are measured routinely, the dose variables of a PD prescription are determined invariably by trial and error in today's standard of care. Such an approach is costly, inefficient, and increases patient burden.

### SUMMARY

This summary is provided to introduce a selection of concepts in a simplified form that are further described below. This summary is not intended to necessarily identify key features or essential features of the present disclosure. The present disclosure may include the following various aspects and embodiments.

In one example, a method of updating a peritoneal dialysis (PD) prescription of a patient is provided. The method comprises: withdrawing a plurality of samples of peritoneal dialysis effluent from the patient's peritoneal cavity periodically during one or more dwell phases of one or more peritoneal dialysis treatment cycles; obtaining sensor measurements of the samples of peritoneal dialysis effluent; determining one or more peritoneal membrane transport parameters based on the sensor measurements; and updating an individualized peritoneal dialysis prescription for the patient, to be used for a later peritoneal dialysis treatment, based on the one or more peritoneal membrane transport parameters.

Alternatively or additionally to any of the examples above, the method can further perform the later peritoneal dialysis treatment for the patient using the updated individualized peritoneal dialysis prescription.

Alternatively or additionally to any of the examples above, withdrawing the plurality of samples comprises withdrawing the plurality of samples during a plurality of different dwell phases of the peritoneal dialysis treatment, and at least one of a dwell duration and a glucose concentration is different for the plurality of different dwell phases.

Alternatively or additionally to any of the examples above, the sensor measurements of the samples of the peritoneal dialysis effluent indicate one or more glucose concentrations and one or more conductivity levels of one or more electrolyte concentrations of the samples of the peritoneal dialysis effluent.

Alternatively or additionally to any of the examples above, the one or more peritoneal membrane transport parameters comprise a glucose transport constant, and determining the glucose transport constant comprises: performing an optimization procedure that fits a volume model for a peritoneal cavity fluid volume to the glucose concentrations from the sensor measurements of the samples of peritoneal dialysis effluent.

Alternatively or additionally to any of the examples above, the volume model is either a fixed volume model represented by the following equation:

$${R}_{Glu}^{D / D 0} ( t ) = \frac{{C}_{d}}{{C}_{d} ( 0 )} = \frac{{C}_{b}}{{C}_{d} ( 0 )} - ( \frac{{C}_{b}}{{C}_{d} ( 0 )} - \frac{{C}_{d} ( 0 )}{{C}_{d} ( 0 )} ) {e}^{- \frac{t}{{τ}_{Glu}}} = \frac{{C}_{b}}{{C}_{d} ( 0 )} + ( 1 - \frac{{C}_{b}}{{C}_{d} ( 0 )} ) {e}^{- \frac{t}{{τ}_{Glu}}} ;$$

or the volume model is a variable volume represented by the following equation:

$${R}_{Glu}^{D / D 0} ( t ) = \frac{{C}_{b}}{{C}_{d} ( 0 )} + \frac{{V}_{Tot}^{PC} ( 0 )}{{V}_{Tot}^{PC} ( t )} ( 1 - \frac{{C}_{b}}{{C}_{d} ( 0 )} ) {e}^{- \frac{t}{{τ}_{glu}}} ;$$

wherein R_{Glu}^{D/D0}(t) is the temporal variation of glucose in PD effluent during the dwell phase, C_{b} is the concentration of glucose in blood, C_{d} is the concentration of glucose in the PD fluid contained in the peritoneal cavity, τ_{Glu} is the glucose transport constant, and V_{Tot}^{PC} is the total volume of fluid in the peritoneal cavity.

Alternatively or additionally to any of the examples above, the optimization procedure utilizes a least squares method.

Alternatively or additionally to any of the examples above, the one or more peritoneal membrane transport parameters comprise a creatine rate constant (τ_{Creat}) and a urea rate constant (τ_{Urea}), which are determined based on the glucose transport constant and universal constants relating the glucose rate constant to the creatine and urea rate constant.

Alternatively or additionally to any of the examples above, wherein the one or more peritoneal membrane transport parameters comprise a rate of fluid absorption from the peritoneal cavity (J_{v_Abs}) and product of an average reflection coefficient (σ_{Av}) and an ultrafiltration (UF) coefficient (k_{UF}), and determining J_{v_Abs}, and the product of σ_{Av} and k_{UF}, comprises: determining a glucose transport constant by performing an optimization procedure that fits a volume model for peritoneal cavity fluid volume to the glucose concentrations from the sensor measurements of the samples of peritoneal dialysis effluent; obtaining UF volumes for the dwell phases where the sensor measurements of the samples of PD effluent are obtained; and determining J_{v_Abs} and a product of σ_{Av} and k_{UF} based on the glucose transport constant, the UF volumes, and the glucose concentrations from the sensor measurements.

Alternatively or additionally to any of the examples above, J_{v_Abs} and the product of σ_{Av} and k_{UF} are determined utilizing the following equation:

$${V}_{Tot}^{PC} ( t ) = {V}_{fill} + {τ}_{glu} \cdot {k}_{UF} \cdot {σ}_{Av} \cdot Δ {P}_{Osm} ( 0 ) \cdot ( 1 - {e}^{- \frac{t}{{τ}_{glu}}} ) - {J}_{v \_ Abs} \cdot t ;$$

wherein V_{Tot}^{PC} is a total volume of fluid in the peritoneal cavity, V_{fill} is a volume of PD fluid supplied during the fill phase, ΔP_{Osm}(0) is an initial transperitoneal differential osmotic pressure, and t is time.

Alternatively or additionally to any of the examples above, withdrawing the plurality of samples of the peritoneal dialysis effluent from the patient's peritoneal cavity comprises providing, by a computing device and to a pump of a peritoneal dialysis system, first instructions to withdraw the plurality of samples, and obtaining the sensor measurements comprises obtaining the sensor measurements by the computing device and in response to providing the first instructions to withdraw the plurality of samples.

Alternatively or additionally to any of the examples above, withdrawing the plurality of samples of the peritoneal dialysis effluent from the patient's peritoneal cavity further comprises: subsequent to providing the first instructions, providing, by the computing device, second instructions to fill the patient's peritoneal cavity with fresh peritoneal dialysis effluent.

Alternatively or additionally to any of the examples above, the peritoneal dialysis system comprises one or more buffer zones in fluid communication with the pump, withdrawing the plurality of samples of the peritoneal dialysis effluent from the patient's peritoneal cavity further comprises: subsequent to providing the first instructions, providing, by the computing device, second instructions to fill the patient's peritoneal cavity with effluent from the one or more buffer zones.

Alternatively or additionally to any of the examples above, the peritoneal dialysis system comprises one or more buffer zones in fluid communication with the pump, and withdrawing the plurality of samples of the peritoneal dialysis effluent from the patient's peritoneal cavity further comprises: subsequent to providing the first instructions, providing, by the computing device, second instructions to fill the patient's peritoneal cavity with effluent from the one or more buffer zones.

Alternatively or additionally to any of the examples above, the individualized peritoneal dialysis prescription indicates a PD fluid glucose concentration, a PD fluid volume, and a dwell time for the patient, and updating the individualized peritoneal dialysis prescription for the patient comprises adjusting at least one of the PD fluid glucose concentration, the PD fluid volume, or the dwell time based on the one or more peritoneal membrane transport parameters.

Alternatively or additionally to any of the examples above, adjusting the at least one of the PD fluid glucose concentration, the PD fluid volume, or the dwell time based on the one or more peritoneal membrane transport parameters comprises: using the one or more peritoneal membrane transport parameters and one or more peritoneal dialysis prescription machine learning (ML)—artificial intelligence (AI) algorithms to determine an adjustment to the PD fluid glucose concentration, the PD fluid volume, or the dwell time.

Alternatively or additionally to any of the examples above, the method can further use the one or more peritoneal membrane transport parameters and one or more peritoneal dialysis failure ML—AI algorithms to determine a predicted time period for a membrane of the patient's peritoneal cavity to fail.

In another example, a peritoneal dialysis (PD) system is provided. The system comprises: a pump configured to be fluidly connected to a PD patient line, wherein the pump is configured to withdraw samples of PD effluent periodically during a dwell phase of a peritoneal treatment for a patient; a PD sensor in fluid communication with the patient line and configured to take sensor measurements of PD effluent entering or exiting the PD patient line; a computing device, programmed to perform a peritoneal dialysis treatment using an individualized peritoneal dialysis prescription, wherein the individualized peritoneal dialysis prescription indicates a PD fluid glucose concentration, a PD fluid volume, and a dwell time for the patient; wherein the computing device is configured to: calculate one or more peritoneal membrane transport parameters based on a plurality of sensor measurements; update the individualized peritoneal dialysis prescription for the patient based on the one or more peritoneal membrane transport parameters; and perform a subsequent peritoneal dialysis treatment for the patient using the updated individualized peritoneal dialysis prescription.

In yet another example, a method of determining a glucose transport constant of a peritoneal membrane of a peritoneal dialysis patient is provided. The method comprises: withdrawing a plurality of samples of peritoneal dialysis effluent from the patient's peritoneal cavity periodically during a dwell phase of a peritoneal dialysis treatment; obtaining, by a sensor device connected to a patient line, a plurality of glucose sensor measurements of the plurality of samples of peritoneal dialysis effluent; and determining the glucose transport constant by performing an optimization procedure that fits a volume model for a peritoneal cavity fluid volume to the glucose sensor measurements.

In yet another example, a method of determining a free water volume of the ultrafiltration for a peritoneal dialysis patient is provided. The method comprises: measuring a conductivity of PD effluent during a drain phase of a PD treatment; measuring a conductivity of fresh PD fluid during a fill phase of the PD treatment; measuring a conductivity of PD effluent one or more times during a dwell phase of the PD treatment; and determining a free water volume of the ultrafiltration based on the conductivity of the PD effluent during the drain phase, the conductivity of the fresh PD fluid during the fill phase, and the conductivity of the PD effluent during the dwell phase.

Alternatively or additionally to any of the examples above, determining the free water volume of the ultrafiltration (V_{FW}^{UF}) utilizes the following equations:

$${V}_{FW}^{UF} = {V}_{Tot}^{UF} - {V}_{Sp}^{UF} ; and$$

$${V}_{Sp}^{UF} = \frac{{V}_{Drain} \cdot {σ}_{Eff} - {V}_{Fill} \cdot {σ}_{PDF}}{{σ}_{pls}}$$

wherein V_{Tot}^{UF} t is a total UF volume, V_{Sp}^{UF} is a small pore ultrafiltrate volume, V_{Drain} is a volume of the fluid drained from the peritoneal cavity, σ_{Eff} is a conductivity of the PD effluent, σ_{PDF} is a conductivity of the fresh PD fluid, and σₚₗₛ is a conductivity of plasma, which is substituted with a conductivity of the PD effluent, σ_{drain}, during the drain phase.

In yet another example, a method of determining a residual cavity volume of a peritoneal dialysis (PD) patient is provided. The method comprises: measuring glucose of a PD effluent during a drain phase to obtained glucose concentration of a residual cavity fluid (C_{Res}); measuring glucose of a fresh PD fluid during a fill phase to obtain glucose concentration of the fresh PD fluid (C_{PDF}); measuring glucose of a mixture of the residual cavity fluid and the fresh PD fluid from the peritoneal cavity (C_{Mix}); and determining the residual cavity volume based on a fill volume (V_{fill}), C_{Res}, C_{PDF}, and C_{Mix}.

Alternatively or additionally to any of the examples above, determining the residual cavity volume (V_{Res}) utilizes the following equation:

$${V}_{Res} = \frac{{V}_{fill} ( {C}_{PDF} - {C}_{Mix} )}{{C}_{Mix} - {C}_{Res}} .$$

Further features and aspects are described in additional detail below with reference to the figures.

### BRIEF DESCRIPTION OF THE DRAWINGS

FIG. 1A is a schematic diagram of an exemplary medical treatment system according to one or more examples of the present application.

FIG. 1B is another schematic diagram of an exemplary medical treatment system according to one or more examples of the present application.

FIG. 2 is a simplified block diagram depicting an exemplary computing environment in accordance with one or more examples of the present application according to one or more examples of the present application.

FIG. 3 is a simplified block diagram of one or more devices or systems within the exemplary environment of FIG. 2 according to one or more examples of the present application.

FIG. 4 is a simplified schematic diagram depicting an exemplary medical treatment system according to one or more examples of the present application.

FIGS. 5A-5D show graphical representations for methods for performing peritoneal dialysis treatment according to one or more examples of the present disclosure.

FIG. 6 is a flowchart of an exemplary process for determining and using peritoneal membrane transport parameters, residual cavity volume, and/or free water component of the ultrafiltration, according to one or more examples of the present application.

FIG. 7 is a flowchart of an exemplary process for determining peritoneal membrane transport parameters, which may be performed as part of the process of FIG. 6, according to one or more examples of the present disclosure.

FIG. 8 is a flowchart of an exemplary process for determining free water volume of ultrafiltrate volume for a peritoneal dialysis patient, according to one or more examples of the present disclosure.

FIG. 9 is a flowchart of an exemplary process for determining the residual cavity volume for a peritoneal dialysis patient, according to one or more examples of the present disclosure.

FIG. 10 is an exemplary plot of D/DO of glucose versus time for fixed and variable volume models of a peritoneal cavity, according to one or more examples of the present disclosure.

FIG. 11 is a plot of temporal variation of D/P ratio of glucose, creatine, and urea versus time during a dwell phase of an exemplary peritoneal dialysis cycle, according to one or more examples of the present disclosure.

FIG. 12 is an exemplary schematic diagram illustrating relevant solute and volume fluxes associated with the peritoneal cavity and passing through the peritoneal membrane, according to one or more examples of the present disclosure.

FIG. 13 is a plot of conductivity versus time for an exemplary peritoneal dialysis treatment, according to one or more examples of the present disclosure.

FIG. 14 is an example plot of glucose and volume versus time for an exemplary peritoneal dialysis treatment, according to one or more examples of the present disclosure.

FIG. 15 is an example plot of membrane transport parameters (k_{UF}·σ_{Av}) versus creatine rate constant τ_{Creat} with an example mapping of peritoneal membrane transport parameters to membrane pathologies, according to one or more examples of the present disclosure.

FIG. 16 is an example plot of membrane transport parameters (k_{UF}·σ_{Av}) and creatine rate constant τ_{Creat} versus time, according to one or more examples of the present disclosure.

FIG. 17 is a flowchart of an exemplary process for determining the peritoneal clearance metric for a peritoneal dialysis patient, according to one or more examples of the present disclosure.

### DETAILED DESCRIPTION

Exemplary embodiments of the present application provide for systems and methods (e.g., automated methods) for determination of peritoneal membrane transport parameters (aPMFA), residual cavity volume (RCV), and/or peritoneal adequacy. For instance, a system may be deployed in the home setting, and may include a non-invasive PD-Effluent Composition Monitor (PD-ECM), which may be attached to the patient line or a line fluidly coupled to the patient line.

During the dwell phase, measurement of several samples of PD effluent enables the rate of glucose dissipation to be established in the individual patient, from which creatinine and urea rate constants may be estimated, without the need for blood sampling. A change in dose variables (e.g., dwell duration and/or glucose concentration) between consecutive cycles combined with appropriate algorithms permits the ultrafiltration coefficient (e.g., fluid transport characteristic of the membrane), fluid absorption from the peritoneal cavity, and average reflection coefficient to be calculated. When the conductivity of PD fluid is measured by the PD-ECM during fill and drain phases, the free water volume of ultrafiltrate may be determined, also without the need for blood sampling. A further set of appropriately timed measurements of glucose concentration during fill, drain and dwell phases, allows the system to determine (e.g., estimate) residual cavity volume, again obviating the need for a blood sample.

In contrast to the traditional trial and error approach mentioned above, examples of the present disclosure allow for a more practical, efficient, and objective approach. For instance, the systems and methods described herein allow for modeling the expected outcome for a given set of dose variables, and a treatment schedule that offers flexibility for the patient. This may use the introduction of appropriate diagnostic methods that enable a broad range of peritoneal cavity and peritoneal membrane transport characteristics, to be determined. To track changes in membrane transport characteristics to guide the prescription revision in a timely manner, such diagnostics may be applied on a frequent basis and/or in the home setting.

The following disclosure relates to methods and systems that perform an automated Peritoneal Membrane Function Assessment (aPMFA), an automated or automated determination of the free water volume of the ultrafiltrate (UF), automated measurement of residual cavity volume (RCV), and/or an automated measurement of peritoneal adequacy (e.g., based on determining a peritoneal clearance metric). One or more aspects of the present disclosure include: a treatment system with the capability to change dose variables between cycles (e.g., glucose concentration and/or dwell duration), a variety of methods for performing timed samples of the PD fluid (fresh and/or effluent), a non-invasive PD-ECM (e.g., a PD-ECM that measures glucose content and/or conductivity) that has the capability to determine measurements (e.g., glucose and/or conductivity), and analytical methods to process the diagnostic data and provide information on peritoneal membrane transport parameters, free water volume of UF, and/or residual cavity volume.

In some examples, embodiments of the present disclosure provide: a device and/or method that performs an assessment of peritoneal membrane function automatically, through the use of a non-invasive PD-ECM, the use of a variety of methods involving a PD treatment system to sample dialysis effluent, and the application of algorithms to determine peritoneal membrane transport parameters, free water volume of UF, residual cavity volume, and/or peritoneal adequacy.

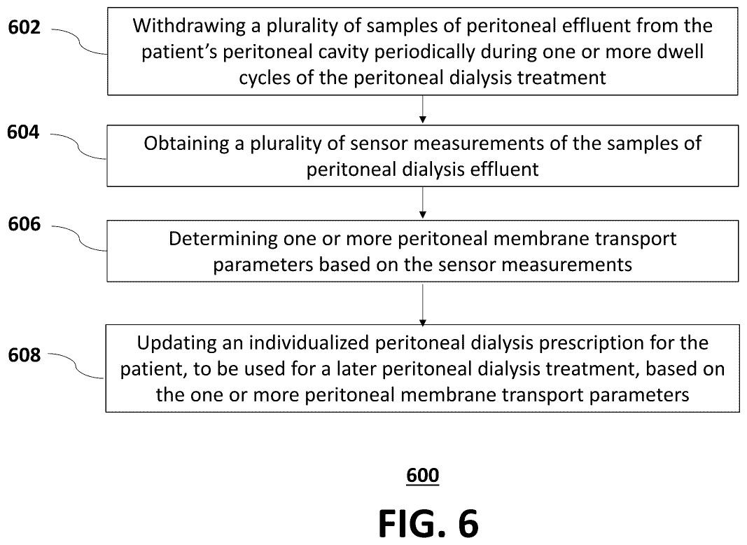

In some variations, PD fluid may be used for the PD treatment for a patient. As used herein, the PD fluid may refer to fresh PD fluid and/or PD effluent. For example, the fresh PD fluid may be PD dialysate that has yet to be introduced to the patient's peritoneal cavity (e.g., the fresh PD fluid that is provided to the patient during the fill phase). The PD effluent may refer to fluid withdrawn from the patient's peritoneal cavity including during the dwell phase (e.g., to be used to determine the peritoneal membrane transport parameters that are described in further detail in FIG. 6 below) and/or the drain phase.

Among other advantages, using examples of the present disclosure allows for reduction to operational cost, improved reliability and accuracy, and significant care burden reduction for both patients and health care professionals. As the systems of the present disclosure lends itself very well to frequent measurement operation, it opens a new gateway to: improved peritoneal membrane diagnostics, enhanced tools for objective PD prescription creation and revision that minimize the patient's glucose exposure, whilst offering more treatment schedule flexibility, prognostic capabilities (e.g., modality transition planning by tracking changes in membrane characteristics), and/or improvements in the management of the PD catheter.

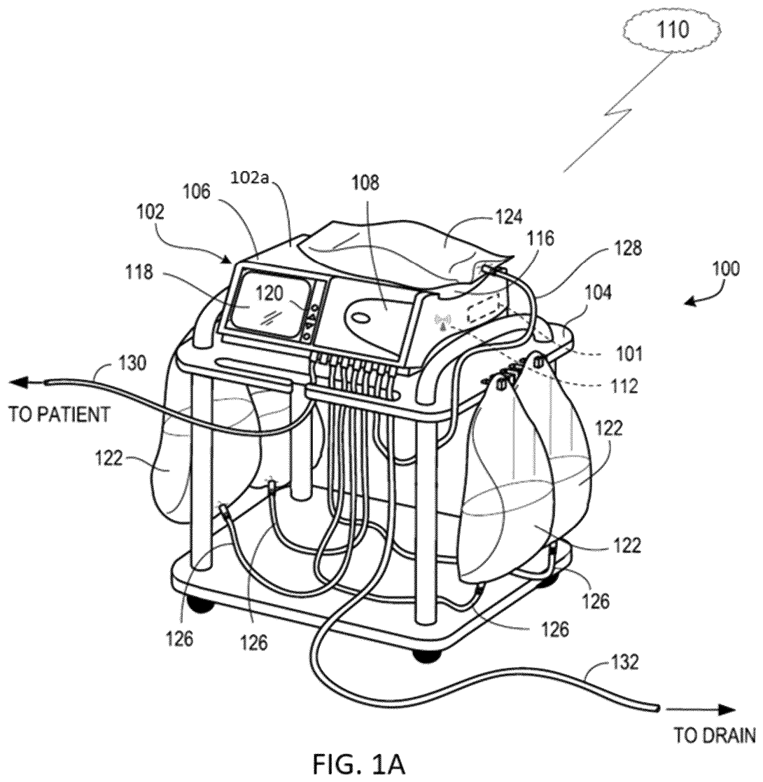

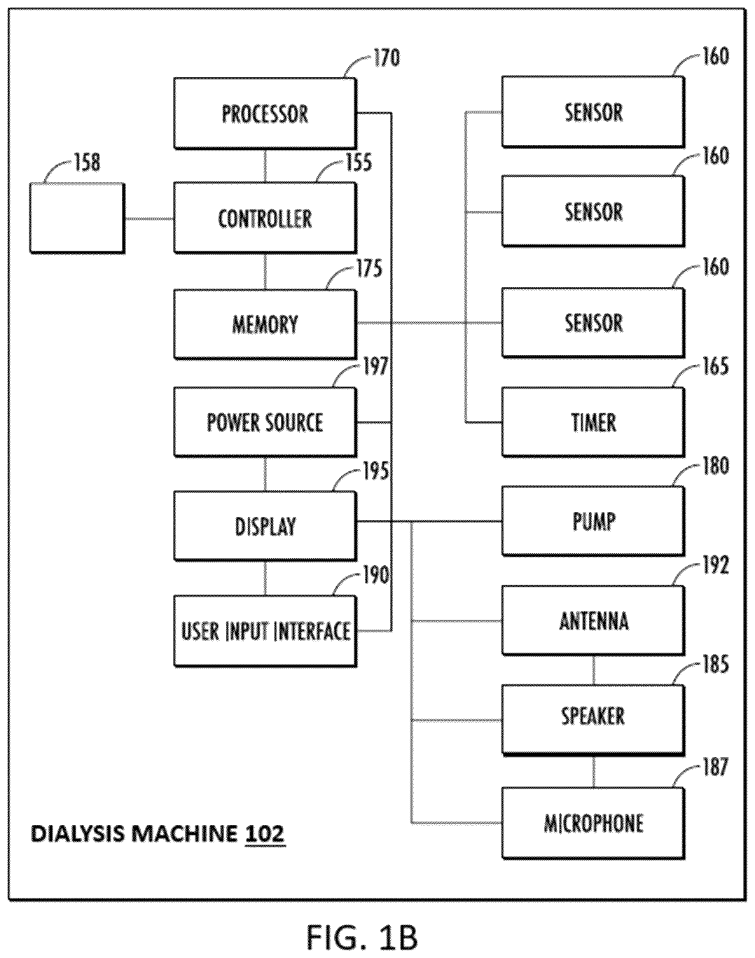

FIGS. 1A and 1B are schematic diagrams of an exemplary medical system (e.g., a dialysis system) according to one or more examples of the present application. By way of example, the medical system shown in FIG. 1A is a peritoneal dialysis system; however, other medical systems are contemplated herein.

In particular, FIG. 1A shows an example of a medical system, implemented as a peritoneal dialysis (PD) system 100, which is configured for use in accordance with an exemplary implementation of the system described herein. In some examples, the PD system 100 may be configured for use at a patient's home (e.g., a home dialysis system). The PD system 100 may include a dialysis machine 102 (e.g. a PD machine, also referred to as a PD cycler), which in some embodiments may be seated on a cart 104. The dialysis machine 102 may include a housing 106, a door 108, and a cartridge interface for contacting a disposable PD cassette, or cartridge, when the cartridge is disposed within a compartment formed between the cartridge interface and the closed door 108. A heater tray 116 may be positioned on top 102a of the housing 106. The heater tray 116 may be any size and shape to accommodate a bag of dialysate (e.g., a 5 liter (L) bag of dialysate). The dialysis machine 102 may also include a user interface such as a touch screen 118 and control panel 120 operable by a user (e.g., a caregiver or a patient) to allow, for example, set up, initiation, and/or termination of a PD treatment.

Dialysate bags 122 may be suspended from the sides of a cart 104, and a heater bag 124 may be positioned in the heater tray 116. Valves may be attached to a bottom portion of the dialysate bags 122 so fluid is drawn out and air delivery is minimized. Dialysate from the dialysate bags 122 may be transferred to the heater bag 124 in batches. For example, a batch of dialysate may be transferred from the dialysate bags 122 to the heater bag 124, where the dialysate is heated by the heating element. When the batch of dialysate has reached a predetermined temperature (e.g., approximately 98°−100° F., 37° C.), the batch of dialysate may be flowed into the patient. The dialysate bags 122 and the heater bag 124 may be connected to the cartridge via dialysate bag lines 126 and a heater bag line 128, respectively. The dialysate bag lines 126 may be used to pass dialysate from dialysate bags 122 to the cartridge during use, and the heater bag line 128 may be used to pass dialysate back and forth between the cartridge and the heater bag 124 during use. In addition, a patient line 130 (e.g., a tubing connected to the patient) and a drain line 132 may be connected to the cartridge. The patient line 130 may be fluidly connected to a patient's peritoneal cavity via a catheter and may be used to pass dialysate back and forth between the cartridge and the patient's peritoneal cavity during use. The drain line 132 may be connected to a drain or drain receptacle and may be used to pass dialysate from the cartridge to the drain or drain receptacle during use.

The touch screen 118 and the control panel 120 may allow a user to input various treatment parameters to the dialysis machine 102 and to otherwise control the dialysis machine 102. In addition, the touch screen 118 may serve as a display. The touch screen 118 may function to provide information to the patient and the operator of the PD system 100. For example, the touch screen 118 may display information related to a dialysis treatment to be applied to the patient, including information related to a prescription. In various embodiments, the control panel 120 may also include audio and video component capabilities, including speakers, microphones and/or cameras.

The dialysis machine 102 may include a processing module 101 that resides inside the dialysis machine 102, the processing module 101 being configured to communicate with the touch screen 118 and the control panel 120. The processing module 101 may be configured to receive data from the touch screen 118 the control panel 120 and sensors, e.g., temperature and pressure sensors, and control the dialysis machine 102 based on the received data. For example, the processing module 101 may adjust the operating parameters of the dialysis machine 102.

The dialysis machine 102 may be configured to connect to a network 110. The connection to network 110 may be via a wireless connection, such as via WIFI or BLUETOOTH, or in some cases a non-wireless connection, as further discussed elsewhere herein. The dialysis machine 102 may include a connection component 112 configured to facilitate the connection to the network 110. The connection component 112 may be a transceiver for wireless connections and/or other signal processor for processing signals transmitted and received over a wired connection. In the case of a wired connection, the connection component 112 may be a port enabling a physical connection to a network component. Other medical devices (e.g., other dialysis machines) or components may be configured to connect to the network 110 and communicate with the dialysis machine 102.

FIG. 1B is a schematic illustration of an exemplary embodiment of a dialysis machine such as, for example, the dialysis machine 102 that is configured for use in accordance with the present disclosure. The machine 102 may be a home dialysis machine, e.g., a PD machine, for performing a dialysis treatment on a patient, and may be included in the system 100 described above. A controller 155, that may be a component of the processing module 101, may automatically control execution of a treatment function during a course of dialysis treatment. The controller 155 may be operatively connected to the sensors 160 and deliver a signal to execute a treatment function or a course of treatment associated with various treatment systems. In some embodiments, a timer 165 may be included for timing triggering of the sensors 160.

In some embodiments, the machine 102 may also include a processor 170, and memory 175, the controller 155, the processor 170, and/or the memory 175, or combinations thereof, that may separately or collectively part of the processing module 101, that may receive signals from the sensor(s) 160 indicating various parameters. Each fluid bag (e.g., the dialysate bags 122) may contain an approximate amount of dialysate, such that “approximate amount” may be defined as a 3 liter (L) fluid bag containing 3000 to 3150 milliliters (mL), a 5 L fluid bag containing 5000 to 5250 mL, and a 6 L fluid bag containing 6000 to 6300 mL. The controller 155 may also detect connection of all fluid bags 122 connected.

Communication between the controller 155 and the treatment system may be bi-directional, whereby the treatment system acknowledges control signals, and/or may provide state information associated with the treatment system and/or requested operations. For example, system state information may include a state associated with specific operations to be executed by the treatment system (e.g., trigger pumps and/or compressors to deliver dialysate and the like) and a status associated with specific operations (e.g., ready to execute, executing, completed, successfully completed, queued for execution, waiting for control signal, and the like).

In some embodiments, the dialysis machine 102 may include at least one pump 180 operatively connected to the controller 155. During a treatment operation, the controller 155 may control the pump 180 for pumping fluid, e.g., fresh and spent dialysate such as the fresh PD fluid and the PD effluent, to and from a patient. For example, the pump 180 may transfer dialysate from the dialysate bag 122 through, for example, a cassette insertable into a port formed in the dialysis machine, to the heating chamber 152 prior to transferring the dialysis to the patient. In an embodiment, the pump 180 may be, for example, a peristaltic pump. The controller 155 may also be operatively connected to a speaker 185 and a microphone 187 disposed in the machine 102. A user input interface 190 may include a combination of hardware and software components that allow the controller 155 to communicate with an external entity, such as a patient, caregiver or other user. These components may be configured to receive information from actions such as physical movement or gestures and verbal intonation. In some embodiments, the components of the user input interface 190 may provide information to external entities. Examples of the components that may be employed within the user input interface 190 include keypads, buttons, microphones, touch screens, gesture recognition devices, display screens, and speakers. The machine 102 may also be wirelessly connectable via an antenna 192 for remote communication that may be a part of the connection component 112. The machine 102 may also include a display 195 and a power source 197.

The sensors 160 may be included for monitoring parameters and may be operatively connected to at least the controller 155, the processor 170, and/or the memory 175, or combinations thereof. The processor 170 may be configured to execute an operating system, which may provide platform services to application software, e.g., for operating the dialysis machine 102. These platform services may include inter-process and network communication, file system management and standard database manipulation. One or more of many operating systems may be used, and examples are not limited to any particular operating system or operating system characteristic.

The memory 175 may include a computer readable and writeable nonvolatile data storage medium configured to store non-transitory instructions and data. In addition, the memory 175 may include a processor memory that stores data during operation of the processor 170. In some examples, the processor memory includes a relatively high performance, volatile, random access memory such as dynamic random-access memory (DRAM), static memory (SRAM), or synchronous DRAM. However, the processor memory may include any device for storing data, such as a non-volatile memory, with sufficient throughput and storage capacity to support the functions described herein. Further, examples are not limited to a particular memory, memory system, or data storage system.

The instructions stored on the memory 175 may include executable programs or other code that may be executed by the processor 170. The instructions may be persistently stored as encoded signals, and the instructions may cause the processor 170 to perform the functions described herein. The memory 175 may include information that is recorded, on or in, the medium, and this information may be processed by the processor 170 during execution of instructions. The memory 175 may also include, for example, specification of data records for user timing requirements, timing for treatment and/or operations, historic sensor information, and the like. The medium may, for example, be optical disk, magnetic disk or flash memory, among others, and may be permanently affixed to, or removable from, the controller 155.

The sensor(s) 160 may include a pressure sensor for monitoring fluid pressure of the machine 102, although the sensors 160 may also include any of a heart rate sensor, a respiration sensor, a temperature sensor, a weight sensor, an air sensor, a video sensor, a thermal imaging sensor, an electroencephalogram sensor, a motion sensor, an audio sensor, an accelerometer, a capacitance sensor, a flow rate sensor, or any other suitable sensor. It is appreciated that the sensors 160 may include sensors with varying sampling rates, including wireless sensors. In some variations, the sensors 160 may include a PD-ECM. For example, the PD-ECM may be included (e.g., coupled to and/or attached to) the patient line 130. During PD treatment, the PD-ECM may obtain sensor measurements (e.g., glucose concentration, temperature, and/or conductivity measurements) and provide them to the controller 155. Additionally, and/or alternatively, the PD-ECM may include one or more processors and/or memory, and may be configured to perform one or more functions, processes, and/or methods such as determining and/or using the peritoneal membrane transport parameters.

The controller 155 may be disposed in the machine 102 or may be coupled to the machine 102 via a communication port or wireless communication links, shown schematically as communication element 158 that may be a part of the connection component 112. According to various examples, the communication element 158 may support a variety of one or more standards and protocols, examples of which include wireless and/or non-wireless communication, such as USB, Wi-Fi, TCP/IP, Ethernet, Bluetooth, among others. As a component disposed within the machine 102, the controller 155 may be operatively connected to any of the sensors 160, the pump 180, and the like. The controller 155 may communicate control signals or triggering voltages to the components of the machine 102. As discussed, exemplary embodiments of the controller 155 may include wireless communication interfaces. The controller 155 may detect remote devices to determine if any remote sensors are available to augment any sensor data being used to evaluate the patient.

It will be appreciated that the medical system depicted in FIGS. 1A and 1B is merely exemplary. The principles discussed herein may be applicable to other medical systems, including various types of peritoneal dialysis systems and techniques (e.g., continuous ambulatory peritoneal dialysis (CAPD) and automated peritoneal dialysis (APD)).

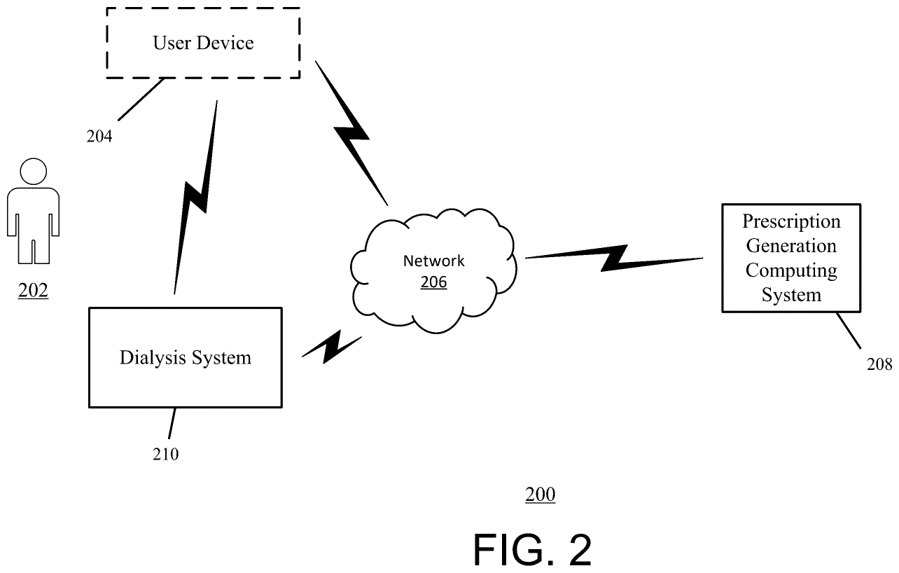

FIG. 2 is a simplified block diagram depicting an exemplary computing environment in accordance with one or more examples of the present application. The environment 200 includes a user (e.g., a patient or individual undergoing peritoneal dialysis treatment) 202, a user device (e.g., mobile device) 204 associated with the user 202, a prescription generation computing system 208, and a dialysis system 210. Although the entities within environment 200 may be described below and/or depicted in the figures as being singular entities, it will be appreciated that the entities and functionalities discussed herein may be implemented by and/or include one or more entities.

The entities within the environment 200 may be in communication with other systems within the environment 200 via the network 206. The network 206 may be a global area network (GAN) such as the Internet, a wide area network (WAN), a local area network (LAN), or any other type of network or combination of networks. The network 206 may provide a wireline, wireless, or a combination of wireline and wireless communication between the entities within the environment 200.

In some instances, one or more entities within environment 200 may be in communication with each other without using the network 206. For instance, the user device 204 and the dialysis system 210 may communicate with each without using the network 206. For example, the dialysis system 210 may be deployed in a home setting (e.g., in the home of the patient 202). As such, the user device 204 and/or the dialysis system 210 may communicate with each other via WI-FI, BLUTOOTH, and/or other communication methods that might not use the network 206.

The dialysis system 210 may be the medical system depicted in FIGS. 1A and 1B (e.g., the PD system 100 and/or the dialysis machine 102) and/or another medical system (e.g., the dialysis system 210 may be or include another type of dialysis and/or peritoneal dialysis machine that performs dialysis treatment). The dialysis system 210 may provide and/or receive information from other entities within the environment 200. For instance, the dialysis system 210 may perform dialysis treatment for a patient 202 based on information received from other entities of environment 200.

The user 202 may operate, own, and/or otherwise be associated with the user device 204 and/or the dialysis system 210. For instance, the user 202 may be a patient that uses the dialysis system 210 to perform dialysis treatment. The user device 204 is denoted in a dashed box to indicate that the user device 204 is optional within the environment 200. When present, the user device 204 is associated with the user 202, and the user 202 may use the user device 204 and/or the dialysis system 210 to perform the dialysis treatment.

The user device 204 may be and/or include, but is not limited to, a desktop, laptop, tablet, computing platforms, mobile device (e.g., smartphone device, or other mobile device), smart watch, an internet of things (IoT) device, or any other type of computing device that generally comprises one or more communication components, one or more processing components, and one or more memory components. The user device 204 may be able to execute software applications managed by, in communication with, and/or otherwise associated with an enterprise organization. Additionally, and/or alternatively, the user device 204 may be configured to operate a web browser. The enterprise organization may be any type of corporation, company, organization, and/or other institution. In some instances, the enterprise organization provides medical services such as dialysis treatment services.

The prescription generation computing device 208 is a computing device and/or system that is associated with the enterprise organization. The prescription generation computing device 208 includes one or more computing devices, computing platforms, systems, servers, and/or other apparatuses capable of performing tasks, functions, and/or other actions for the enterprise organization. In some instances, the prescription generation computing device 208 may, for example, generate one or more initial dialysis prescriptions (e.g., PD prescriptions) for the patient 202. The prescription generation computing device 208 may provide the generated initial dialysis prescriptions to the user device 204 and/or the dialysis system 210.

The prescription generation computing device 208 may be implemented using one or more computing platforms, devices, servers, and/or apparatuses. In some variations, the prescription generation computing device 208 may be implemented as engines, software functions, and/or applications. In other words, the functionalities of the prescription generation computing device 208 may be implemented as software instructions stored in storage (e.g., memory) and executed by one or more processors.

In operation, the environment 200 may be configured for automated peritoneal dialysis (APD) and/or continuous ambulatory peritoneal dialysis (CAPD). For APD, the patient 202 may set up the dialysis system 210 (e.g., the PD cycler shown in FIG. 1), and the dialysis system 210 may perform dialysis treatment automatically (e.g., the dialysis system 210 such as the PD cycler shown in FIG. 1A may perform dialysis treatment overnight). In other words, for APD, a dialysis machine may be used to deliver and drain the PD fluid (e.g., the fresh PD fluid and/or the PD effluent) automatically, with minimal human intervention.

For CAPD, the patient 202 might not use a machine (e.g., the PD cycler shown in FIG. 1). Instead, the patient 202 may perform exchanges by hand by connecting their patient line to a drain bag to drain PD effluent from their peritoneal cavity and connect a fresh PD fluid bag (e.g., a PD fluid source) to their catheter to fill their peritoneal cavity with fresh PD fluid. Gravity may be used to facilitate filling of peritoneal cavity with fresh PD fluid by hanging the fresh PD fluid bag above the elevation of the patient's peritoneal cavity. Once a dwell time is reached, the patient may use gravity to facilitate draining of the PD effluent from the peritoneal cavity to a drain bag by placement of the drain bag below the elevation of the patient's peritoneal cavity. For CAPD, a sensor may be attached to the patient line to take measurements of the fresh PD fluid during fill, the PD effluent during drain, and/or PD effluent samples withdrawn during the dwell, which may be used to determine the peritoneal membrane transport parameters (aPMFA), free water volume of UF, and/or residual cavity volume. In some instances, the user device 202 may include a sensor that is configured to obtain sensor measurements. The user device 202 and/or another computing entity may use the sensor measurements to determine the aPMFA and/or the RCV. The user device 202 may further provide alerts to the patient 202 such as when the dwell time has been reached and/or when the patient 202 should take the sensor measurements.

In some instances, the environment 200 may use a hybrid approach between the CAPD and the APD. For instance, a pump or a gravity feed may be used to facilitate delivering and/or draining the dialysate for the patient 202. The user device 202 and/or another sensor may be used to obtain sensor measurements during the dwell time, and may use the sensor measurements for determining the aPMFA and/or the RCV.

It will be appreciated that the exemplary environment depicted in FIG. 2 is merely an example, and that the principles discussed herein may also be applicable to other situations or examples.

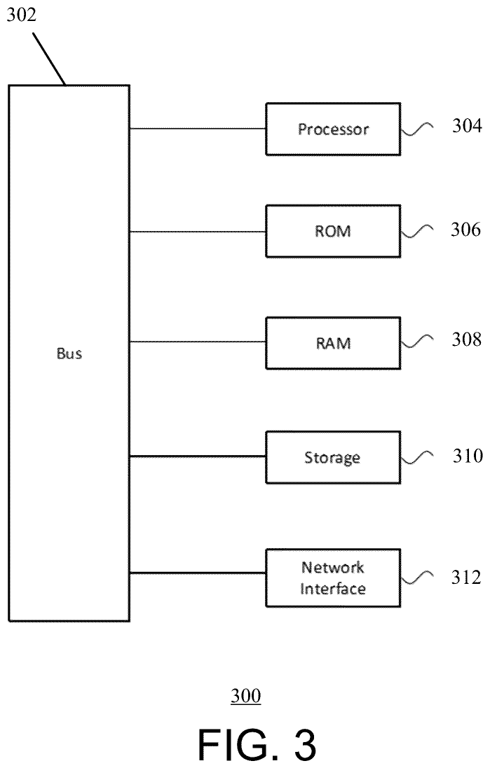

FIG. 3 is a simplified block diagram of one or more devices or systems within the exemplary environment of FIG. 2 according to one or more examples of the present application. For instance, the device/system 300 may be the user device 204 and/or the prescription generation computing system 208. The device/system 300 includes a processor 304, such as a central processing unit (CPU), controller, and/or logic, that executes computer executable instructions for performing the functions, processes, and/or methods described herein. In some examples, the computer executable instructions are locally stored and accessed from a non-transitory computer readable medium, such as storage 310, which may be a hard drive or flash drive. Read Only Memory (ROM) 306 includes computer executable instructions for initializing the processor 304, while the random-access memory (RAM) 308 is the main memory for loading and processing instructions executed by the processor 304. The network interface 312 may connect to a wired network or cellular network and to a local area network or wide area network, such as the network 206. The device/system 300 may also include a bus 302 that connects the processor 304, ROM 306, RAM 308, storage 310, and/or the network interface 312. The components within the device/system 300 may use the bus 302 to communicate with each other. The components within the device/system 300 are merely exemplary and might not be inclusive of every component, server, device, computing platform, sensor, and/or computing apparatus within the device/system 300. Additionally, and/or alternatively, the device/system 300 may further include components that might not be included within every entity of environment 200.

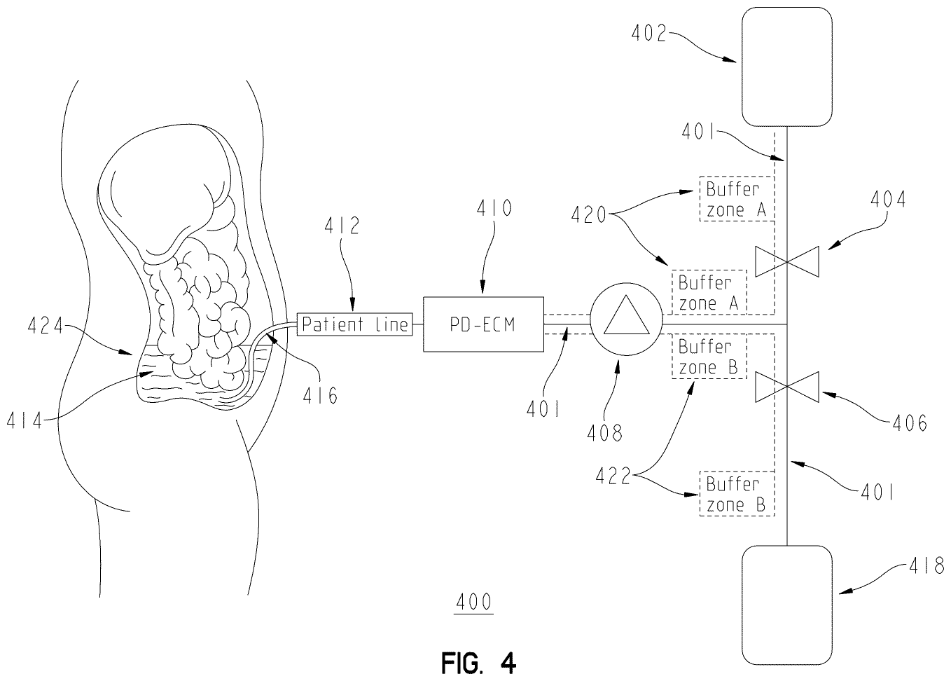

FIG. 4A is a simplified schematic diagram depicting an exemplary medical treatment system according to one or more examples of the present application. It will be appreciated that the medical treatment system 400 depicted in FIG. 4A is merely an example, and that the principles discussed herein may also be applicable to other situations or examples. For instance, the system 400 may be used for APD, but as mentioned above, the system and process described herein may also be used for CAPD and/or a hybrid approach of both APD and CAPD. For example, in some embodiments, gravity can be used to facilitate filling and draining of fluid (e.g., PD effluent) from the peritoneal cavity in place of pump 408. The system 400 may include, among other aspects, a PD fluid source 402 (e.g., a PD fluid storage, a PD fluid container, a PD fluid bag such as the dialysate bag 122 of FIG. 1, and/or another element that is capable of storing the fresh PD fluid), a first and second clamps 404 and 406, a pump 408, a PD-ECM 410, a PD effluent drain 418 (e.g., a drain bag, a drain line such as the drain line 132, and/or another element that is capable of storing or removing the PD effluent after the PD effluent is drained from the patient's peritoneal cavity 414), buffer zones 420 and 422, and fluid tubing 401 fluidly connecting two or more components of system 400. System 400 may be configured to fluidly connect to a patient line 412 that is connected to a patient catheter 416 (e.g., surgically installed in the patient's peritoneal cavity 414).

The medical treatment system shown in FIG. 4A may be used for automated peritoneal membrane function assessment to determine peritoneal membrane transport parameters, to determine residual cavity volume, to determine free water component of UF, and/or to determine peritoneal adequacy. The two clamps 404, 406 and the pump 408 may be implemented in a variety of ways, including the all-in-one dedicated cassette system, found in many modern pump assisted APD cyclers (e.g., dialysis machine 102). The two clamps 404, 406 can work alternately to control fill and drain phases. During a fill phase, fresh PD fluid from the PD fluid source 402 is supplied into the peritoneal cavity 414 via the patient line 412, assisted either by gravity and/or mechanical pump (e.g., the pump 408). During a drain phase, the peritoneal cavity 414 may be emptied under the influence of gravity or by mechanical pump (e.g., the pump 408). The PD-ECM 410 may be connected to the patient line 412 during the fill phase, dwell phase, and drain phase so that both fresh PD fluid and/or PD effluent may be measured for subsequent analysis. For instance, referring to FIG. 1A, the PD-ECM 410 may be attached, coupled, connected, and/or otherwise associated with the patient line 130, and may be used to obtain sensor measurements of PD fluid flowing through the patient line 130.

The PD fluid source 402 may include dialysate (e.g., fresh PD fluid) that may be selected based on a patient's dialysis prescription. In some instances, the dialysis prescription may include information including, but not limited to, a PD fluid glucose concentration, a PD fluid volume, a dwell time, a cycle frequency, and/or total number of cycles. The PD fluid glucose concentration is the amount of glucose concentration that is within the fresh PD fluid. For example, PD fluid bags are available having different glucose concentrations (e.g., 1.5%, 2.3%, 2.5%, 4.25%). The PD fluid volume is the fill volume or the amount of liquid (e.g., the volume) that is administered to the patient for the PD treatment. For example, PD fluid bags of different volumes (e.g., 1.5 liters, 2 liters, 2.5 liters, 3 liters, 5 liters, or 6 liters) are available depending on the patient and PD dialysis technique. The dwell time is the amount of time or duration that the dialysate stays within the patient 202 (e.g., within the patient's peritoneal cavity 414 during the dwell phase). Typically, the PD prescription may be one of the standard glucose concentrations available for PD fluid bags. In some instances, the PD prescription may identify a non-standard glucose concentration, and the control system may be configured to generate a PD fluid solution having this non-standard concentration. The control system may accomplish this in one or more ways. For example, one option is mixing fresh PD fluid solutions of different glucose concentration (e.g., 1.5% and 2.3%), another option is diluting the PD fluid solution to reduce the glucose concentration, and another way may be spiking the PD fluid solution with a small concentrated glucose solution to increase the overall glucose concentration.

Traditionally, an initial dialysate prescription may be determined for a patient, and that initial dialysate prescription may be used for weeks, months, or even years. However, the patient's response to their PD treatments may and likely will change over time, and as such, updated dialysate prescriptions for the patient may be required to provide the optimal treatment for the patient. For example, the membrane function of the patient (e.g., the peritoneum membrane that lines the inside of the abdomen and pelvis) that is used for PD may change over time. For instance, when a patient initially starts PD (e.g., by using the initial dialysate prescription), the peritoneum may function efficiently. In other words, when dialysate (e.g., fresh PD fluid) is provided into the patient, the peritoneum may help facilitate and/or act as a filter to remove waste product from blood. However, over time, the peritoneum functionality may deteriorate (e.g., based on morphological alteration of the peritoneum when it is used as a dialysis membrane), and might not operate as efficiently or effectively as the patient continues their PD treatment. Eventually, the peritoneum functionality may lead to functional failure, and the patient may have to move onwards from PD treatment to another type of treatment such as hemodialysis (HD) treatment. As such, by automatically updating the dialysate prescriptions regularly, which is described below, this may assist in avoiding, minimizing, and/or delaying the deterioration of the peritoneum and allow for the patient to continue utilizing PD therapy for longer. For instance, once the dialysate reaches the body, the peritoneum acts as a filter and uses diffusion and/or convective transport. After a certain amount of time, the dialysate reaches equilibrium in the body and actually reverses, which is noted by a pressure decrease. Therefore, treatment at that time becomes less efficient, and is the optimal time for draining. By using updated PD prescriptions (e.g., with updated dwell times, glucose concentrations, and/or fill volume), the dialysate may be moved closer to equilibrium, which causes less stress on the peritoneum. Additionally, in some instances, updating a patient's PD prescription regularly also enables the PD prescription to utilize a lower glucose concentration of PD fluid, which may also reduce stress on the peritoneum. By causing the peritoneum less stress, this may slow the deterioration of the peritoneum membrane function and extend the duration a patient may be able to stay on PD therapy for longer.

A PD treatment cycle may include three phases—a fill phase, a dwell phase, and a drain phase. During the fill phase, the PD fluid is provided from the PD fluid source 402 into the patient's peritoneal cavity 414. The pump 408 and/or another mechanism (e.g., gravity feed and/or the clamps 404, 406) may be used to supply the fresh PD fluid to the peritoneal cavity 414. For instance, the control system (e.g., the controller 155, the processor 170, and/or the PD-ECM 410) may provide instructions to the pump 408 to facilitate the delivery of the fresh PD fluid to the peritoneal cavity 414 via the patient line 412. The patient line 412 is connected to a catheter 416 that is surgically placed in the patient's abdominal cavity. In some instances, PD-ECM 410 may obtain sensor measurements of the PD fluid during the fill phase as it is delivered to the patient's peritoneal cavity.

After the fresh PD fluid is delivered, the dwell phase begins. The recommended duration of the dwell phase (i.e., dwell time) may be set by the PD prescription. During the dwell phase the PD fluid resides inside the peritoneal cavity 414 and absorbs waste and extra fluid from the patient's body through the patient's peritoneal membrane which acts as a semi-permeable membrane filter by allowing the passage of water and solute.

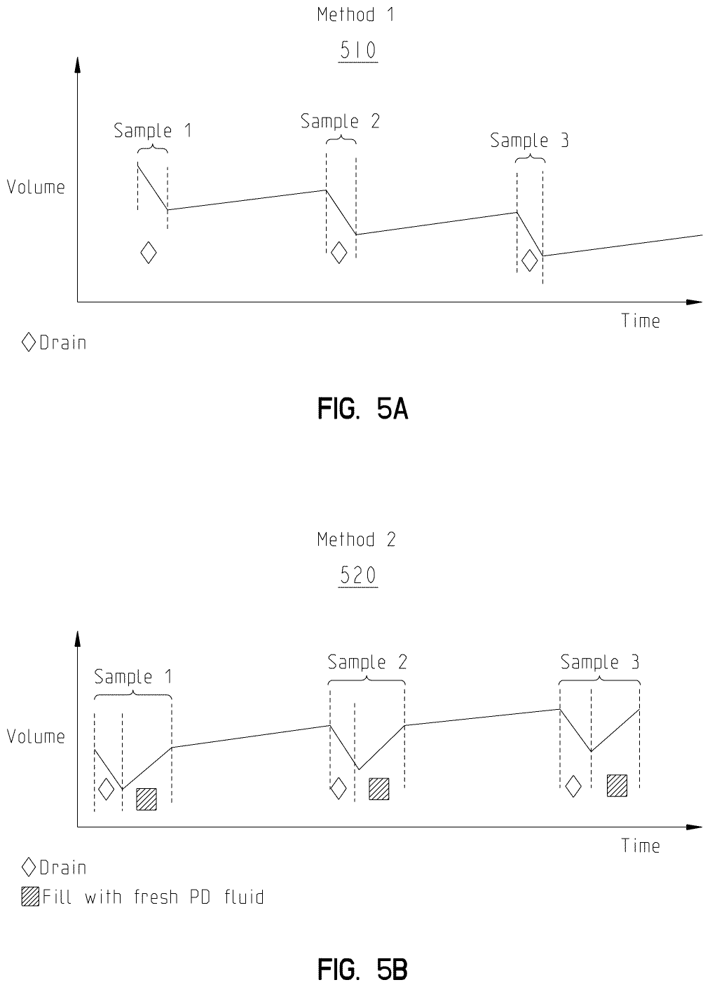

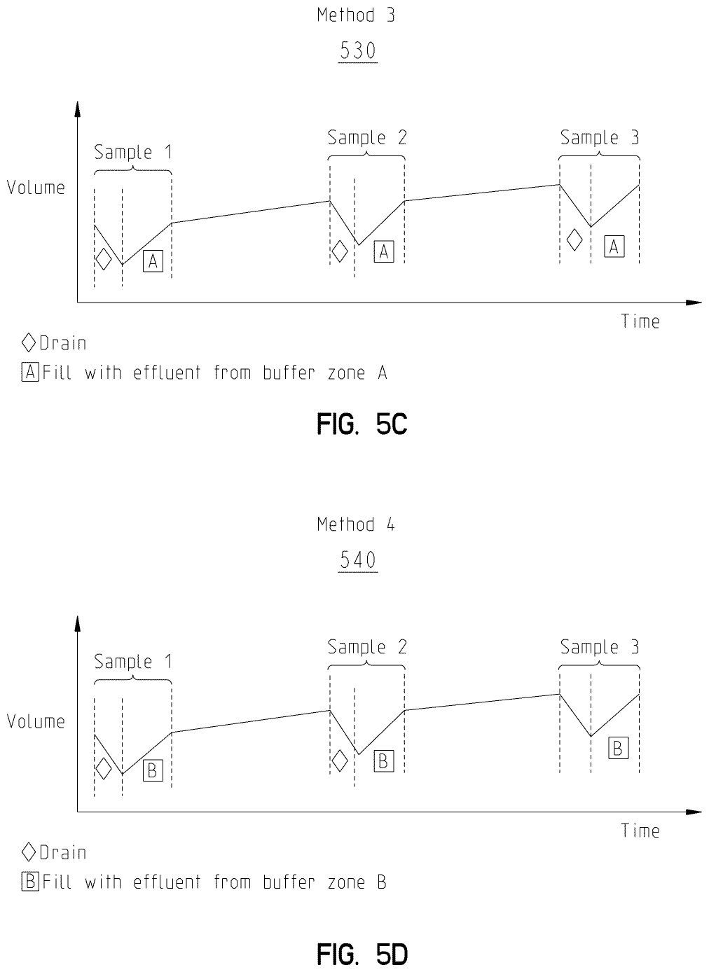

During the dwell phase, the control system may be configured to use the pump 408 and/or other mechanisms to withdraw samples of PD effluent from the patient's peritoneal cavity 414. For example, the control system may use the patient line 412 and the pump 408 to withdraw samples of PD effluent from the patient's peritoneal cavity 414 thereby passing by the PD-ECM, enabling PD-ECM to collect sensor measurements associated with the samples. For instance, the PD-ECM 410 may include one or more sensors, and may use the one or more sensors to obtain sensor measurements from the withdrawn samples during the dwell phase. For example, as will be explained below, the control system (e.g., the PD-ECM 410 and/or another entity) may use the sensor measurements (e.g., glucose concentration and/or conductivity of the withdrawn samples) to perform one or more tasks including, but not limited to, updating the PD prescription. During the dwell phase, which may last hours, the control system may periodically and/or intermittently retrieve a plurality of PD effluent samples (e.g., three or more samples) from the patient's peritoneal cavity 414, and obtain sensor measurements for each of the samples. The frequency, timing, and/or variability of obtaining the PD effluent samples during the dwell phase is described in further detail below, for example, in reference to FIGS. 5A-5D.

Subsequently, the drain phase may start after the dwell phase. During the drain phase, the pump 408 and/or another mechanism may be used to drain the PD fluid/waste (e.g., the PD effluent) from the patient's peritoneal cavity 414 into the PD effluent drain 418. In some instances, PD-ECM 410 may obtain sensor measurements of the PD effluent during the drain phase.

For each PD treatment, a patient may undergo multiple cycles (fill phase, dwell phase, and drain phase). For instance, the patient may undergo two or three, or sometimes four or five cycles consecutive cycles per PD treatment. By using the methods and/or process described below, and updating the PD prescription on a regular and ongoing basis, this may provide the patient with more flexibility as the patients may be able to adjust or select a PD treatment schedule (e.g., frequency of PD treatments, length of cycles, and/or number of cycles) that best fits their life and schedule while still achieve the desired clearance of waste and fluids.

The PD fluid sample measurements during the dwell phase may be performed in several different ways. For example, the pump 408 may be configured to pump fluid in both directions through the patient line 412 (e.g., delivery of the PD fluid to the cavity 414 and withdrawing the PD effluent from the cavity 414). Similarly, gravity can also be used to facilitate the flow of PD fluid/effluent in both directions through the patient line. During the dwell phase, the PD-ECM 410 may obtain sensor measurements (e.g., glucose measurements and/or conductivity measurements from each sample) as PD effluent is withdrawn from the peritoneal cavity 414. In some instances, the pump 408 may return the withdrawn sample PD effluent to the peritoneal cavity after obtaining the sensor measurements, and/or fresh PD fluid may be supplied (e.g., from the PD fluid source 402). For instance, using the Buffer zones A and/or B 420, 422, the pump 408 may withdraw and store the withdrawn PD effluent into one or more of the Buffer zones 420 and/or 422. Additionally, and/or alternatively, the pump 408 may withdraw the PD effluent and remove it completely by providing it to the PD effluent drain 418. This process may be repeated multiple times (e.g., three or more times) over the course of the dwell phase of a cycle of the PD treatment. Whether the sample of PD effluent is returned to the peritoneal cavity 414, sent to PD effluent drain 418, or fresh PD fluid is supplied may be adjustable and can be changed for each sample depending on the PD prescription/and or sensor measurements in order to maintain desired peritoneal cavity fluid volume and concentration.

FIGS. 5A-5D show graphical representations of PD fluid volume in the peritoneal cavity versus time for several different methods 510-540 for sampling of PD effluent during a dwell phase of a PD treatment cycle.

For example, referring to FIG. 5A and the first method 510, at different times during the dwell phase, the control system may withdraw samples (e.g., samples of PD effluent) such as sample 1, sample 2, and sample 3. The control system may use the pump 408 to drain the PD effluent from the peritoneal cavity 414. For first method 510, all three of the withdrawn samples are sent to drain (e.g., PD effluent drain 418), which results in the PD fluid volume in the peritoneal cavity 414 decreasing during each sampling period. In between sampling periods, the fluid volume in the peritoneal cavity is shown gradually increasing, illustrating how typically the PD fluid draws excess fluid into the peritoneal cavity 414 of the patient. For first method 510, the overall volume decreases over the duration of the dwell.

Referring to FIG. 5B, for the second method 520, the control system may withdraw sample 1, sample 2, and sample 3, and also refill with fresh PD fluid (e.g., from the PD fluid source 402) after one or more of the samplings. For method 520 shown in FIG. 5B, fresh PD fluid is supplied after sample 1, sample 2, and sample 3; however, in some instances, fresh PD fluid may be supplied after all, after any one of the samples, and/or after any two of the samples. Although this uses more resources in terms of PD fluid, it may be advantageous in instances where there is a need to enhance solute clearance or ultrafiltration volume. For second method 520, the overall volume increases over the duration of the dwell.

Referring to FIG. 5C, for the third method 530, the control system may withdraw sample 1, sample 2, and sample 3, by withdrawing a portion of the PD effluent to buffer zone A 420 and then returning it to the patient's peritoneal cavity 414. For third method 530 shown in FIG. 5C, each sample is withdrawn and returned; however, in some instances, the PD effluent sample may be returned after only any one of the samples and/or after any two of the samples. For third method 530, the overall volume increases over the duration of the dwell.

Referring to FIG. 5D, for the fourth method 540, the control system may withdraw sample 1, sample 2, and sample 3, but instead of using the buffer zone A 420 in method 3 530, this fourth method 540 may use the buffer zone B 422. For the fourth method 540 shown in FIG. 5D, the withdrawn PD effluent is returned from buffer zone B after all three samples; however, in some instances, the PD effluent may be returned after only any one or any two of the samples.

In some implementations, buffer zones 420 and 422 of the third and fourth methods 530 and 540, indicated in FIG. 4A, may be used for more than just holding a withdrawn sample of PD effluent during samples. These buffer zones also allow a small volume of PD effluent to be moved back and forth several times to enable improved mixing in the peritoneal cavity 414. This helps to ensure that the PD-ECM 410 reads a composition PD effluent that is representative of the composition in the peritoneal cavity 414. Use of the buffer zones 420 and 424 in this manner, is a method that is best suited to pump assisted APD cyclers. An advantage of second method 520, third method 530, and fourth method 540 is that in principle, an unlimited number of PD effluent samples may be read by the PD-ECM 410 during a dwell, as there is no net change in cavity volume due to the sampling procedure.

In some instances, the control system may utilize a combination of methods 1, 2, 3, and/or 4. For example, for sample 1, the PD effluent may be withdrawn and then returned from buffer zone 420 or 422, for sample 2, the PD effluent may be sent to drain 418, and for sample 3, fresh PD fluid may be returned to the patient's peritoneal cavity rather than the withdrawn sample of PD effluent. In some instances, the control system may carry out any of the different actions (e.g., send PD effluent sample to drain 418, fill with fresh PD fluid, or refill with withdrawn PD effluent from one of the buffer zones 420/422).

In some instances, the control system (e.g., the PD-ECM 410) may determine a plurality of times during the dwell cycle. For example, in some variations, the control system may obtain one or more samples during the first hour as samples during this time frame may be more interesting than samples later on. For instance, the control system may use the pump 408 to obtain a first sample right after the dwell phase begins (e.g., immediately after the fill phase). In some examples, the control system may obtain five samples in the first hour, and may obtain additional samples at a later time (e.g., during the third hour and/or the fourth hour of the dwell phase).

In some variations, the control system may receive input from PD prescription and/or a care provider indicating when to take the samples during the dwell cycle. In other variations, the control system may use one or more artificial intelligence (AI)—machine learning (ML) algorithms or models to determine the timing for obtaining the samples.

After obtaining the sensor measurements associated with the samples, the control system may perform one or more functions and/or tasks such as determining one or more peritoneal membrane transport parameters, updating the peritoneal dialysis prescription (e.g., the initial PD prescription described above), and/or other tasks (e.g., determining the RCV and/or free water component of the UF).

In various implementations of the present disclosure, programmed instructions and/or one or programmed algorithms that may be stored in memory and run by a processor, can include the various methods and process (e.g., process 600, 700, 800, 900) discussed further herein.

For instance, FIG. 6 is a flowchart of an exemplary process 600 for determining and using peritoneal membrane transport parameters according to one or more examples of the present application. It will be recognized that any of the following steps may be performed in any suitable order, and that the process 600 may be performed in any suitable environment. Process 600 may be performed, for example, by PD system 100, controller 155, dialysis system 210, medical treatment system 400, or other devices and systems, described herein. The descriptions, illustrations, and processes of FIG. 6 are merely exemplary and the process 600 may use other descriptions, illustrations, and processes for determining and using peritoneal membrane transport parameters.

In operation, at step 602, process 600 can include withdrawing a plurality of samples of peritoneal dialysis effluent from a patient's peritoneal cavity periodically during one or more dwell cycles of their peritoneal dialysis treatment. For example, the control system (e.g., the controller 155 and/or the processor 170 of FIG. 1A and/or the PD-ECM 410 of FIG. 4) may provide one or more instructions to the pump 408 and/or another mechanism to withdraw a sample of PD effluent from the patient. For example, referring to FIG. 4, based on the instructions, the pump 408 may be configured to withdraw samples from the peritoneal cavity 414 via the patient line 412. Based on, for example, methods 510-540 from FIGS. 5A-5D, or combination thereof, the pump 408 may pump the PD effluent from the cavity 414 into the buffers 420 and/or 422 and/or the PD effluent drain 418. In other words, in some examples (e.g., the first and second methods 510 and 520), based on the instructions, the pump 408 may pump the PD effluent into the PD effluent drain 418. Additionally, and/or alternatively, the control system may provide instructions for the pump to deliver additional fluid (e.g., the second method 520 for filling with fresh PD fluid) from the PD fluid source 402 into the patient's cavity 414.

In some variations (e.g., the third and fourth methods 530 and 540), based on the instructions, the pump 408 may pump the PD effluent from the cavity 414 into the buffer zones 420 and/or 422. Then, the control system may provide further instructions, and based on the further instructions, the pump 408 may provide a small volume of PD effluent from the buffer zones 420 and 422 to be moved back and forth several times to enable improved mixing in the cavity 414. As mentioned above, this may help to ensure that the PD-ECM 410 reads a composition PD effluent that is representative of the composition in the peritoneal cavity 414.

In some examples, as mentioned above, CAPD may be the PD technique used for the PD treatment. In such examples, the control system (e.g., a user device such as user device 204 of FIG. 2) may withdraw samples by providing prompts or alerts to the user 202. Based on the prompts or alerts, the PD effluent may be withdrawn from the patient's cavity via a patient line. Then, the user device 204, PD-ECM 410, and/or another device (e.g., device that comprises one or more sensors) may obtain sensor measurements associated with the PD effluent withdrawn from the patient's peritoneal cavity 414.

In some instances, the control system may be programmed with instructions to create batches of dialysate based on a patient's current PD prescription. For instance, as mentioned above, the PD prescription may indicate one or more dwell durations, glucose concentrations, and/or fill volume, which are described above. The control system may use the PD prescription to create a batch of dialysate and store the batch of dialysate in the PD fluid source 402. For instance, based on the glucose concentrations and/or the fill volume, the control system may create the batch of fresh PD fluid (e.g., the dialysate). Then, as mentioned above, the control system may initiate the PD treatment, which may include three phases for each cycle. In other instances, the user may use dialysate bags (e.g., bags 122) for the PD treatment. Each of the dialysate bags may include a different glucose concentration, volume, and/or other metrics that are described above. The PD prescription may indicate a dialysate bag for the patient to use for the PD treatment.

In some examples, each cycle may include drain phase, a fill phase, and a dwell phase. After the fill phase is complete (e.g., the fresh PD fluid is delivered to the patient's cavity 414), step 602 may begin. For instance, during the dwell phase (e.g., a dwell cycle within the PD treatment), the control system may obtain a plurality of samples of PD effluent from the cavity 414 such as by using the pump 408. The control system may obtain a plurality of samples for each dwell cycle (e.g., a first dwell cycle or phase associated with a first cycle of the PD treatment, a second dwell cycle or phase associated with a second cycle of the PD treatment, and so on). For instance, for each PD cycle, the control system may obtain a plurality of samples (e.g., three, four, five, or more samples) of PD effluent.

At step 604, the control system obtains a plurality of sensor measurements of the samples of peritoneal dialysis effluent. For example, after withdrawing each sample, the control system may obtain sensor measurements associated with each sample, such as glucose concentrations, conductivity measurements, and temperature. For instance, as mentioned above, the PD-ECM 410 may include one or more sensors that are configured to obtain the sensor measurements (e.g., glucose concentrations and/or conductivity measurements,) from the samples. As mentioned above, for each dwell cycle, the control system may withdraw a plurality of samples, and may obtain sensor measurements for each of the samples. In some implementations, step 604 may also include obtaining sensor measurements of the PD fluid during the fill phase and/or the drain phase. For example, sensor measurements for the fresh PD fluid may be obtained during the fill phase while measurements of the PD effluent may also be obtained during the drain phase.

In some instances, such as when using CAPD, the control system (e.g., the user device or PD-ECM) may obtain the sensor measurements for the withdrawn samples using one or more of its sensors. Additionally, and/or alternatively, the control system (e.g., the user device) may instruct another entity (e.g., a computing device comprising one or more sensors) to obtain the sensor measurements for the withdrawn samples.

Process 600, at step 606 can include determining one or more peritoneal membrane transport parameters based on the plurality of sensor measurements. Process 700 described further herein, is one example of how step 606 may be performed.

Process 600, at step 608 can include updating an individualized peritoneal dialysis prescription for the patient, to be used for a subsequent peritoneal dialysis treatment, based on the one or more peritoneal membrane transport parameters. For instance, using the determined peritoneal membrane transport parameters, the control system may provide an update to the patient's individual peritoneal dialysis prescription (e.g., the PD prescription from the prescription generation computing system 208). For example, the control system may adjust, modify, alter, and/or otherwise change the initial PD prescription including, but not limited to, the initial glucose concentration, dwell time, fill volume, number of cycles, and/or frequency of treatment. In some implementations, at step 608, rather than the control system automatically updating the patient's dialysis prescription, the control system may provide a recommendation for an update to the individualized peritoneal dialysis prescription for the patient. For example, the recommendation may be reviewed by a care provider, and the care provider may then implement the update to the dialysis prescription if appropriate. In another example, some recommendations may be reviewable by the patient (e.g., change to dwell time), and the patient may implement the update to the dialysis prescription. In some implementations, the control system may be configured such that if the update to the patient's dialysis prescription falls within preset acceptable ranges the update may be automatically implemented by the control system, but if the update to the dialysis prescription falls outside the present acceptable ranges, then it requires review and approval by a care provider prior to implementation.

Additionally, and/or alternatively, after each PD treatment and/or each cycle of the PD treatment, the control system may continue to update the peritoneal dialysis prescription for the patient. For example, after an initial update of the patient's PD prescription, during a subsequent PD treatment, the control system may obtain further sensor measurements during the dwell phase. Based on the further sensor measurements, the control system may determine updated peritoneal membrane transport parameters and provide or recommend another update to the PD prescription (e.g., the dwell time of the initial PD prescription may be reduced after each PD treatment based on the sensor measurements).

In some examples, the updated dialysis prescription may indicate a new dialysate bag to use for the subsequent PD treatment. For example, the updated dialysis prescription may indicate to use a different dialysate bag (e.g., a 2 L dialysate bag whereas the initial dialysis prescription may indicate to use a 3 L dialysate bag for the patient) and/or use a different dwell time (e.g., change the dwell time from 4 hours to 3 hours). In other examples, the control system may be configured to instruct a device or machine to create a dialysate to be stored in the PD fluid source 402. In such examples, the control system may use the initial dialysis prescription to create the PD dialysate for use in the PD treatment. Then, after step 608, the control system may use the updated dialysis prescription to create a new batch of PD dialysate based on the updated dialysis prescription. The control system may further automatically initiate the drain phase at a different time (e.g., based on the dwell time changing from 4 hours to 3 hours).

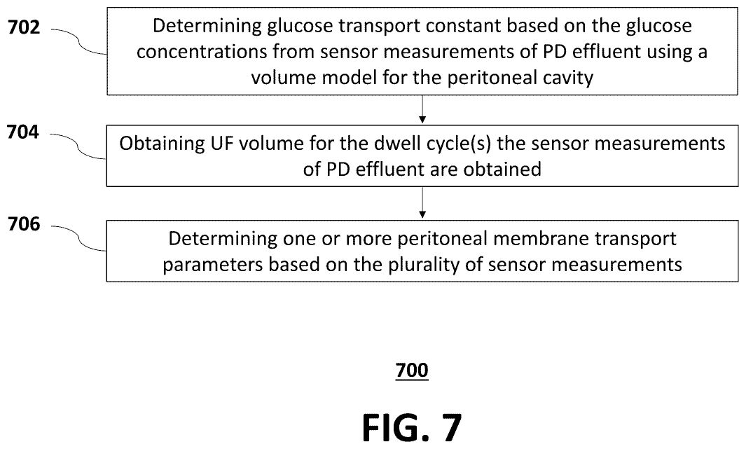

Process 700 shows the steps for determining one or more peritoneal membrane transport parameters based on the plurality of sensor measurements, according to one or more implementations of the present disclosure. For example, process 700 may be utilized to perform step 606 as shown in FIG. 6. Process 700 may be performed, for example, by PD system 100, controller 155, dialysis system 210, medical treatment system 400, or other devices and systems, described herein. The descriptions, illustrations, and processes of FIG. 7 are merely exemplary and the process 700 may use other descriptions, illustrations, and processes for determining peritoneal membrane transport parameters.

Before getting into the details of the steps of process 700, a bit of additional information on what happens during the dwell phase will first be provided to help aid in the explanation of process 700. During the dwell phase of a PD cycle, solutes such as creatinine, urea and glucose pass across the peritoneal membrane. This process is driven by the concentration gradient between the patient's blood plasma and dialysate contained in the peritoneal cavity. Thus, the ratio of the concentrations in the plasma and dialysate undergo change during the dwell phase, but given sufficient time, an equilibrium is established.

As PD is largely a continuous process, the plasma concentration of a given solute is considered to remain constant. However, in order to make quantitative analysis of membrane transport, it may be necessary to obtain the value of the plasma concentration of a solute. Aspects of the present disclosure, including processes 600 and 700, circumvent the need to withdraw a blood sample, instead enabling plasma glucose to be estimated by analysis of glucose in PD effluent. This can enable peritoneal membrane function assessment to be conducted as often as desired in the comfort of the patient's home, without the need for a blood sample.

The estimation of blood plasma glucose involves tracking the temporal variation of the ‘D/D0 ratio’, R_{Glu}^{D/D0}(t) of glucose in PD effluent during the dwell phase. The relevant analytic method may include, for example, first approximating the transport kinetics of glucose across the peritoneal membrane using, for example, the following first order fixed volume model:

$${V}_{Av}^{PC} \frac{{dC}_{d}}{dt} = {k}_{mtac} ( {C}_{b} - {C}_{d} ) \qquad Eq . 1$$

where C_{b} is the concentration of glucose in blood, C_{d} is the concentration of glucose in the PD fluid contained in the peritoneal cavity, V_{Av}^{PC} is the average volume of fluid in the peritoneal cavity during the dwell, and K_{mtac} is the mass transfer area coefficient of the peritoneal cavity wall with respect to glucose. In some implementations of the present disclosure, V_{Av}^{PC} may be considered a constant.

Eq. 1 may be solved to yield:

$${C}_{d} ( t ) = {C}_{b} - ( {C}_{b} - {C}_{d} ( 0 ) ) {e}^{- \frac{t}{{τ}_{Glu}}} = {C}_{b} + ( {C}_{d} ( 0 ) - {C}_{b} ) {e}^{- \frac{t}{{τ}_{Glu}}} \qquad Eq . 2$$

where

$$\frac{1}{{τ}_{Glu}} = \frac{{k}_{mtac}}{{V}_{Av}^{PC}}$$

and C_{d}(0) is the initial concentration of glucose in the peritoneal cavity.

Dividing Eq. 2 throughout by C_{d}(0) yields:

$${R}_{Glu}^{D / D 0} ( t ) = \frac{{C}_{d}}{{C}_{d} ( 0 )} = \frac{{C}_{b}}{{C}_{d} ( 0 )} - ( \frac{{C}_{b}}{{C}_{d} ( 0 )} - \frac{{C}_{d} ( 0 )}{{C}_{d} ( 0 )} ) {e}^{- \frac{t}{{τ}_{Glu}}} = \frac{{C}_{b}}{{C}_{d} ( 0 )} + ( 1 - \frac{{C}_{b}}{{C}_{d} ( 0 )} ) {e}^{- \frac{t}{{τ}_{Glu}}} \qquad Eq . 3$$

In some implementations of the present disclosure, a variable volume model for V_{Av}^{PC} may be utilized to account for the change in volume due to the ultrafiltration process. The differential form of Eq. 1 may be modified as:

$$\frac{d \{ {C}_{d} {V}_{Tot}^{PC} \}}{dt} = {K}_{mtac} ( {C}_{b} - {C}_{d} ) + {Q}_{uf \_ net} {C}_{b} \qquad Eq . 4$$

where V_{Tot}^{PC} is the total volume of fluid in the peritoneal cavity, Q_{uf_net} represents the NET ultrafiltration (net UF), which is regarded as the total ultrafiltration rate minus the lymphatic flow rate. In this case, the net UF operates on the blood side. The first term on the right-hand side of Eq. 4 represents the diffusive transport of solute while the second term accounts for the convective transport owing to net UF.

Solution of Eq. 4 yields:

$${V}_{Tot}^{PC} ( t ) ( {C}_{b} - {C}_{d} ( t ) ) = {V}_{Tot}^{PC} ( 0 ) [ {C}_{b} - {C}_{d} ( 0 ) ] {e}^{- \frac{t}{{τ}_{glu}}} \qquad Eq . 5$$

Dividing Eq. 5 throughout by the initial glucose concentration C_{d}(0) yields:

$${R}_{Glu}^{D / D 0} ( t ) = \frac{{C}_{b}}{{C}_{d} ( 0 )} + \frac{{V}_{Tot}^{PC} ( 0 )}{{V}_{Tot}^{PC} ( t )} ( 1 - \frac{{C}_{b}}{{C}_{d} ( 0 )} ) {e}^{- \frac{t}{{τ}_{glu}}} \qquad Eq . 6$$

To illustrate the difference in behavior of Eq. 3 (fixed volume model) and Eq. 6 (variable volume model), the temporal variation of the cavity volume V_{Tot}^{PC} (t) due to ultrafiltration is taken into account. This may be approximated by an expression of the form:

$${V}_{Tot}^{PC} ( t ) = {V}_{Fill} + {V}_{uf \_ Max} ( 1 - {e}^{- k \cdot t} ) - {Q}_{Lymph} \cdot tdt \qquad Eq . 7$$

where k is a rate constant related to fluid transport, not to be confused with the mass transfer area coefficient of glucose and Q_{Lymph} is a fixed lymphatic flow rate.

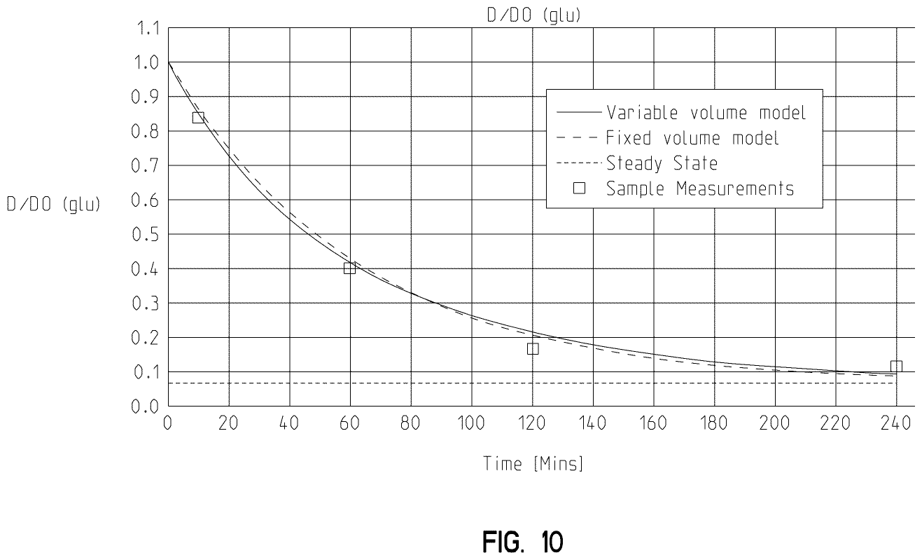

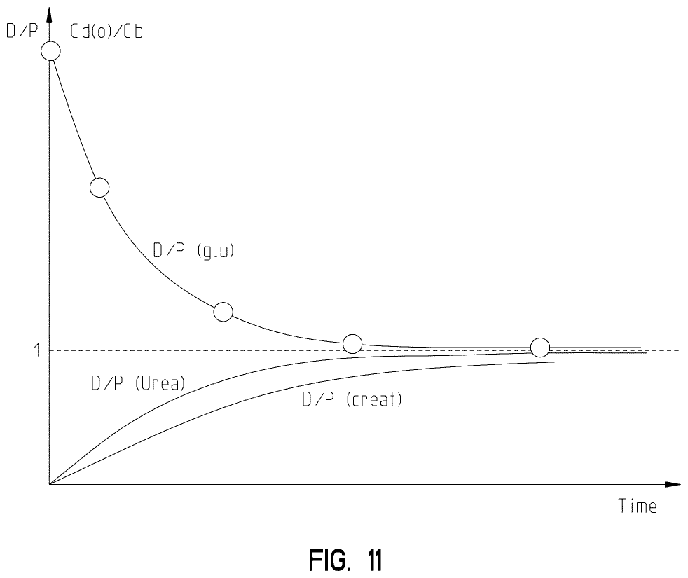

FIG. 10 is a graph of D/D0 (glu) versus time with plots illustrating the behavior of the D/D0 ratio for fixed volume model (Eq. 3) and variable volume model (Eq. 6) of the peritoneal cavity expressions. As illustrated by FIG. 10, the fixed volume model plot and variable mode plot are very similar. Thus, while the variable volume model (Eq. 6) may be more rigorous, utilizing the variable volume model rather than the fixed volume model may have little practical utility in most cases, and thus in most cases the fixed volume model of Eq. 3 will usually be sufficient. The variable volume model (Eq. 6) may be reserved in rare cases, for example, where the UF volume and Fill Volume are similar in magnitude.

Regardless of whether a fixed volume or variable volume model is utilized, the steady state conditions can be of particular utility. For an infinite dwell, the steady state (ss) value achieved may be an expression of the form:

$${R}_{Glu}^{D / D 0} ( ss ) = \frac{{C}_{b}}{{C}_{d} ( 0 )} \qquad Eq . 8$$

Now referring to process 700, process 700 at step 702 can include determining glucose transport constant (τ_{Glu}) and blood glucose (C_{b}) based the glucose concentration measurements of PD effluent (e.g., obtained by step 604 of process 600), using a volume model for the peritoneal cavity. Step 702 may also include determining an estimate of the blood glucose (C_{b}) while determining glucose transport constant. FIG. 10 includes four plotted example glucose concentration measurements from a dwell phase. Determining the glucose transport constant and/or blood glucose may be performed, for example, by an optimization procedure that fits the volume model (e.g., Eq. 3 or Eq. 6) to the PD effluent glucose sample measurements (e.g., four plotted sample measurement in FIG. 10). The least squares method is one example of a statistical procedure that may be used to fit the volume model to the set of PD effluent sample glucose sample measurements. Other optimization procedures may be utilized. From step 702, glucose transport constant/coefficient (t_{Glu}) may be identified and an estimate of the blood glucose (C_{b}) may be obtained, without the need for actual blood testing. Repeating this procedure over successive cycles enables the accuracy of blood glucose estimation to be improved as well enabling the means to track variation in blood glucose on a regular basis.

Process 700, at step 704 can include obtaining the ultrafiltrate volume for the dwell cycle(s) the sensor measurements of the PD effluent samples are performed. For example, UF volume may be calculated based on the weight difference between the fill and drain PD fluid volumes for a cycle. In some implementations, other techniques may be utilized to measure the ultrafiltrate volume.

Process 700, at step 706 can include determining additional membrane transport parameters, based on the UF volume measurements, glucose transport constant/coefficient (τ_{Glu}), and sensor measurements of PD effluent samples. These additional membrane transport parameters may include, for example, average reflection coefficient (σ_{Av}), UF coefficient (k_{UF}), and rate of fluid absorption from the peritoneal cavity (J_{v_Abs}).

Determining the rate of glucose dissipation for an individual patient from the PD effluent samples during the dwell, can also enable creatinine and urea rate constants to be estimated, without the need for blood sampling. For example, dividing Eq. 2 by C_{b} leads to a model of the temporal variation of the D/P ratio. In the case of glucose this is:

$${R}_{Glu}^{D / P} ( t ) = 1 + ( \frac{{C}_{d} ( 0 )}{{C}_{b}} - 1 ) {e}^{- \frac{t}{{τ}_{Glu}}} \qquad Eq . 9$$

The glucose rate constant, τ_{Glu} has the same meaning regardless which of the ratios D/P or D/D0 are of interest. In the case of creatinine and urea, if the initial dialysate concentrations C_{d}(0) are assumed to be zero, then the resulting temporal variation of the corresponding D/P ratios are:

$${R}_{Creat}^{D / P} ( t ) = 1 - {e}^{- \frac{t}{{τ}_{Creat}}} \qquad Eq . 10$$

$${R}_{Urea}^{D / P} ( t ) = 1 - {e}^{- \frac{t}{{τ}_{Urea}}} \qquad Eq . 11$$

The nature of the relationships given by Eq. 9, Eq. 10, Eq. 11 is illustrated by FIG. 11, in which the temporal variation of D/P ratio of Glucose, Creatinine and Urea is shown.

A small inaccessible residual volume is usually present in the peritoneal cavity between successive PD cycles. While this effect may be taken into account when fitting a suitably modified D/P model to measured values of D/P, such model fitting does not apply as the PD-ECM 410 does not measure urea or creatinine. Instead, τ_{Glu} can be used as the basis to obtain the rate constants of creatinine and urea, τ_{Creat} and τ_{Urea}.

During the dwell phase, creatinine and urea pass through the same system of membrane pores as glucose, mainly the small pores. Therefore, in a given patient, the only difference in the kinetics of creatinine, urea and glucose relates to the relative difference of their hydrodynamic sizes. Hence:

$${τ}_{Creat} = {k}_{gc} \cdot {τ}_{Glu} \qquad Eq . 12$$

$${τ}_{Urea} = {k}_{gu} \cdot {τ}_{Glu} \qquad Eq . 13$$

k_{gc} and k_{gc} are considered universal constants relating the glucose rate constant to the creatinine and urea rate constants respectively. The values of k_{gc} and k_{gc} may be determined from any PD cohort where glucose, creatinine and urea have been measured by conventional membrane assessment methods such as the Peritoneal Equilibration Test (PET).

Now turning to the ultrafiltration coefficient (k_{UF}). The UF coefficient of the peritoneal membrane describes the flow rate of ultrafiltrate developed for a given differential driving pressure across the membrane. The UF coefficient is sometimes referred to as the ‘hydraulic conductance due to glucose’ as glucose is the primary source of the driving pressure. As a membrane transport parameter, the UF coefficient is important for prediction of the UF volume to be expected for the prevailing dose variables of the PD prescription.

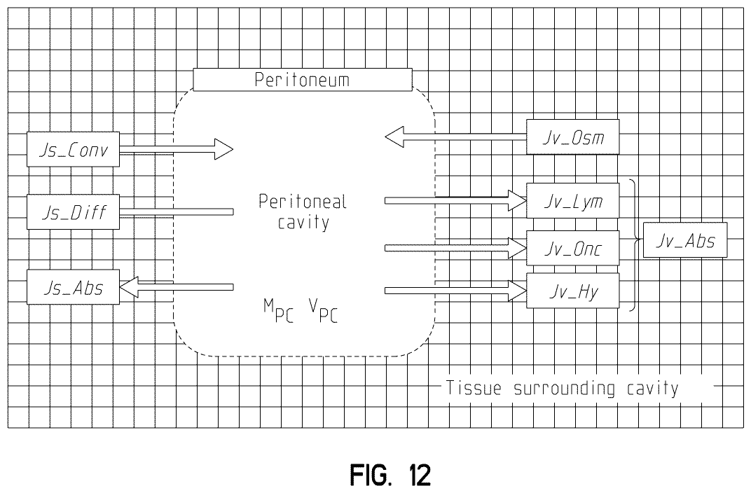

The determination of the UF coefficient may use the application of a suitable 1st principles model. In some instances, some model simplifications may be made so that the model has practical utility in terms of variables that are known or can be measured. FIG. 12 depicts the relevant solute and volume fluxes as seen from the peritoneal cavity passing through the peritoneum. For example, this includes depicting the solute (glucose) and volume fluxes across the peritoneal membrane. In this case the direction of the solute fluxes refers to the osmotically active crystalloids comprised in the peritoneal cavity fluid such as glucose, creatinine, urea, and electrolytes. The solute and volume fluxes, as shown in FIG. 19, vary with time and are defined in Table 1 below.

TABLE 1
Solute and volume flux definitions

| Variable | Description |
|---|---|
| Jₛ_{_{—}}_{Diff} | Diffusive solute flux caused by the concentration gradient |
| | across the peritoneal membrane |
| Jₛ_{_{—}}_{Conv} | Convective solute flux caused by ultrafiltration |
| Jₛ_{_{—}}_{Abs} | Overall solute flux arising from absorption from the |
| | peritoneal cavity |
| Jᵥ_{_{—}}_{Abs} | Overall absorption volume flux, lumping together all |
| | absorption pathways |
| Jᵥ_{_{—}}_{Osm} | Volume flux due to the crystalloid osmotic pressure gradient |
| Jᵥ_{_{—}}_{Onc} | Volume flux component of absorption due to differential |
| | colloid oncotic pressure |
| Jᵥ_{_{—}}_{Hy} | Volume flux component of absorption due to differential |
| | hydrostatic pressure |
| Jᵥ_{_{—}}_{Lym} | Volume flux component of absorption due to direct drainage |
| | of the peritoneal cavity by the lymphatics (lymphatic stomata) |

Using the above variable set, the relevant differential equations may be developed. The rate of change of volume in the peritoneal cavity may be stated represented as:

$${J}_{v \_ Net} = \frac{d}{dt} \{ {V}_{Tot}^{PC} \} = {J}_{v \_ Osm} - {J}_{v \_ Abs} \qquad Eq . 14$$

The absorption flux J_{v_Abs} is comprised of three volume fluxes arising from differential hydrostatic pressure, differential oncotic pressure, and direct lymphatic drainage from the peritoneal cavity. The overall osmotic volume flux, J_{v_Osm} can be determined from the contributions of different crystalloids in the peritoneal cavity and surrounding interstitial space. Via the Van't Hoff relationship then represented as:

$${J}_{v \_ Osm} = {k}_{UF} \cdot RT \cdot {σ}_{Av} \cdot ( {C}_{Osm}^{PC} - {C}_{Osm}^{Inst} ) \qquad Eq . 15$$

where k_{UF} is the overall hydraulic conductance as seen ‘looking out’ from the walls of the peritoneal cavity, RT is the product of the Gas constant and Temperature, C_{Osm}^{PC} and C_{Osm}^{Inst} are aggregated osmolar concentrations of the crystalloids in the peritoneal cavity and the interstitial fluid respectively (e.g., C_{Osm}^{PC} varies with time while C_{Osm}^{Inst} is considered constant for all practical purposes), σ_{Av} is the average reflection coefficient, capturing the relative contributions of large, small and ultrasmall pores, considering all crystalloids.

The rate of change of mass in the peritoneal cavity is dependent on the product of concentration of the m^{th} crystalloid and the volume of the peritoneal cavity. Therefore:

$$\frac{d}{dt} \{ {M}_{PC 〈 m 〉} \} = {V}_{PC}^{Tot} \cdot \frac{d}{dt} \{ {C}_{Osm}^{PC} \} + {C}_{Osm}^{PC} \cdot \frac{d}{dt} \{ {V}_{PC}^{Tot} \} = {J}_{s \_ Conv} - {J}_{s \_ Diff} - {J}_{s \_ Abs} \qquad Eq . 16$$

The convective solute flux from the interstitial fluid may be represented as:

$${J}_{s \_ Conv} = {J}_{v \_ Osm} \cdot {C}_{Osm}^{PC} \qquad Eq . 17$$

While the solute flux due to absorption from the peritoneal cavity may be represented as:

$${J}_{s \_ Abs} = {J}_{v \_ Abs} \cdot {C}_{Osm}^{PC} \qquad Eq . 18$$

The diffusive solute flux caused by the osmotic gradient developed across the membrane may be represented as:

$${J}_{s \_ Diff} = {K}_{mtac} \cdot ( {C}_{Osm}^{PC} - {C}_{Osm}^{Inst} ) \qquad Eq . 19$$

where K_{mtac} is the aggregate mass transfer area coefficient (MTAC) of osmotically active crystalloids.

Consequently, Eq. 14 and 15 may combined to yield:

$${J}_{v \_ Net} = \frac{d}{dt} \{ {V}_{Tot}^{PC} \} = {k}_{UF} \cdot RT \cdot {σ}_{Av} ( {C}_{Osm}^{PC} - {C}_{Osm}^{Inst} ) - {J}_{v}^{Abs} \qquad Eq . 20$$

Substituting Eqs. 17-19 into Eq. 16 yields:

$${V}_{PC}^{Tot} \cdot \frac{d}{dt} \{ {C}_{Osm}^{PC} \} + {C}_{Osm}^{PC} \cdot {J}_{v \_ Net} = {J}_{v \_ Osm} \cdot {C}_{Osm}^{Inst} - {K}_{mtac} \cdot ( {C}_{Osm}^{PC} - {C}_{Osm}^{Inst} ) - {J}_{v \_ Abs} \cdot {C}_{Osm}^{PC} \qquad Eq . 21$$

Substituting Eq. 14 and re-arranging, then leads to:

$$\frac{d}{dt} \{ {C}_{Osm}^{PC} \} = - \frac{( {J}_{v \_ Osm} + {K}_{mtac} ) \cdot ( {C}_{Osm}^{PC} - {C}_{Osm}^{Inst} )}{{V}_{Tot}^{PC}} \qquad Eq . 22$$

As V_{Tot}^{PC} undergoes temporal variation with ultrafiltration during the dwell, Eq. 22 is a non-linear differential equation. This may be solved together with the coupled differential equation Eq. 14 by numerical integration. However, the temporal variation of glucose in the peritoneal cavity may be approximated with a simplified differential equation. The approach adopted characterizes only diffusive transport of glucose (as the primary osmotic agent) across the membrane and assumes a fixed cavity volume. In this case:

$$\frac{d}{dt} \{ {C}_{Osm}^{PC} ( t ) \} \approx - \frac{{K}_{mtac} \cdot ( {C}_{Osm}^{PC} ( t ) - {C}_{Osm}^{Inst} )}{{V}_{Tot}^{PC}} \qquad Eq . 23$$

Integration of Eq. 23 leads to the result:

$${C}_{Osm}^{PC} ( t ) - {C}_{Osm}^{Inst} = Δ {C}_{Osm}^{tp} ( 0 ) \cdot {e}^{- t / {τ}_{Glu}} \qquad Eq . 24$$

$$where$$

$$Δ {C}_{Osm}^{tp} ( 0 ) = {C}_{Osm}^{PC} ( 0 ) - {C}_{Osm}^{Inst} \qquad Eq . 25$$

$$and$$

$${τ}_{Glu} = \frac{{V}_{Tot}^{PC}}{{K}_{mtac}} \qquad Eq . 26$$

ΔC_{Osm}^{tp}(0) is the initial transperitoneal concentration difference of osmotically active particles, i.e., between the peritoneal dialysis fluid in the cavity and the extracellular fluid in the interstitium. The time constant t_{glu} expresses the ratio of the mass transfer area coefficient of glucose K_{mtac} to the cavity volume V_{Tot}^{PC}. Although both K_{mtac} and V_{Tot}^{PC} vary with time, their ratio is considered largely constant. Therefore Eq. 24 may be of particular relevance in the practical setting as neither K_{mtac} nor V_{PC} need to be known.

Substituting Eq. 24 into Eq. 20 yields the following differential equation:

$${J}_{v}^{Net} = \frac{d}{dt} \{ {V}_{PC}^{Tot} \} = {k}_{UF} \cdot RT \cdot {σ}_{Av} \cdot Δ {C}_{Osm}^{tp} ( 0 ) {e}^{- t / {τ}_{glu}} - {J}_{v\_Abs} \qquad Eq . 27$$

The temporal variation of volume in the peritoneal cavity may now be found by integration of Eq. 27:

$${V}_{Tot}^{PC} = {k}_{UF} \cdot RT \cdot {σ}_{Av} \cdot Δ {C}_{Osm}^{tp} ( 0 ) \int {e}^{- t / {τ}_{glu}} - {J}_{v \_ Abs} dt \qquad Eq . 28$$

which leads to

$$Eq . 29$$

$${V}_{Tot}^{PC} ( t ) = {V}_{fill} + {τ}_{glu} \cdot {k}_{UF} \cdot {σ}_{Av} \cdot RT \cdot Δ {C}_{Osm}^{tp} ( 0 ) \cdot ( 1 - {e}^{- \frac{t}{{τ}_{glu}}} ) - {J}_{v \_ Abs} \cdot t$$

In fresh PD fluid (PDF) the number of osmolarity is largely determined largely by glucose, while dissociated ions make a minor contribution. Opposing the osmolarity of the PDF is the osmolarity of extracellular fluid, OsmExtc which may be assumed as 300 mOsm/L in a typical subject. The initial transperitoneal differential osmotic pressure is thus:

$$Δ {P}_{Osm} ( 0 ) = ( {Osm}_{PDF} - {Osm}_{Extc} ) \cdot RT \qquad Eq . 30$$

Substituting Eq. 30 into Eq. 29 results in:

$${V}_{Tot}^{PC} ( t ) = {V}_{fill} + {τ}_{glu} \cdot {k}_{UF} \cdot {σ}_{Av} \cdot Δ {P}_{Osm} ( 0 ) \cdot ( 1 - {e}^{- \frac{t}{{τ}_{glu}}} ) - {J}_{v \_ Abs} \cdot t \qquad Eq . 31$$

Referring back to the steps of process 700, step 706 may include determining one or more peritoneal membrane transport parameters. For example, based on the determined glucose transport constant (e.g., from step 702), the UF volume (e.g., from step 704), and other know or measured values (e.g., PD effluent, glucose concentration, V_{fill}, t) associated with the dwell cycles, a first estimate of membrane transport parameters (e.g., σ_{Av}·k_{UF} and J_{v_Abs}) may be calculated based on optimization modeling of Eq. 31. Process 700 may be repeated for many consecutive PD treatments, which can enable additional accuracy in the determination of the membrane transport parameters.

Without the glucose measurements of the PD effluent during the dwell cycle(s), it would take many treatment cycles for which the PD prescription (e.g., dwell duration and/or the glucose concentration) is changed in order determine membrane transport parameters (e.g., glucose transport constant (τ_{Glu}), product of average reflection coefficient (σ_{Av}) and UF coefficient (k_{UF}), and Rate of fluid absorption from the peritoneal cavity (J_{v_Abs}). Such a lengthy and complex approach would be inconvenient and burdensome to the patient, furthermore it is less sensitive to transient changes in the membrane transport parameters, for example, which may be caused by a peritonitis event.

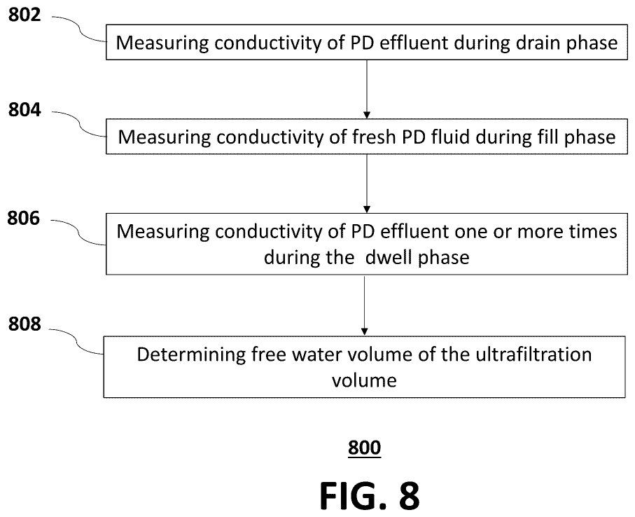

As described herein, in addition to enabling automated determination of membrane transport parameters, methods and system described herein can enable automated determination of the free water volume of the ultrafiltration volume. For instance, FIG. 8 is a flowchart of an exemplary process 800 for determining a free water volume of the ultrafiltration volume, according to one or more examples of the present disclosure. It will be recognized that any of the following steps may be performed in any suitable order, and that the process 800 may be performed in any suitable environment. Process 800 may be performed, for example, by PD system 100, controller 155, dialysis system 210, medical treatment system 400, or other devices and systems described herein. The descriptions, illustrations, and processes of FIG. 8 are merely exemplary and the process 800 may use other descriptions, illustrations, and processes for determining and using peritoneal membrane transport parameters.

Before getting into the details of the steps of process 800, a bit of additional information will be provided to assist in the explanation. In assessment of the fluid transport across the peritoneal membrane, it can be beneficial to know the contribution of ultrafiltrate derived through the aquaporin channels (ultra-small pores). In a healthy peritoneal membrane, water passing through the ultrasmall pores (aquaporin channels) makes a major contribution to the overall UF volume. But this can reduce significantly with increasingly impaired membrane function.

Aquaporin function may be assessed by analysis of the PD effluent sodium content from timed samples. Sodium is sieved by aquaporins (reflection coefficient of 1), and the sodium passing through the very few large pores in the peritoneal membrane is negligible. Therefore, only the small pores of the membrane (of the 3-pore system) are relevant for sodium transport. The mass of sodium that passes small pores is equivalent to the difference in the mass of sodium between the drained fluid and the instilled fluid (PDF filled). Hence, this may be represented as follows:

$${V}_{Sp}^{UF} \cdot {C}_{Na}^{pls} = {V}_{Drain} \cdot {C}_{Na}^{Eff} - {V}_{Fill} \cdot {C}_{Na}^{PDF} \qquad Eq . 32$$

$$Therefore ,$$

$${V}_{Sp}^{UF} = \frac{{V}_{Drain} \cdot {C}_{Eff}^{Na} - {V}_{Fill} \cdot {C}_{PDF}^{Na}}{{C}_{pls}^{Na}} \qquad Eq . 33$$

where V_{Sp}^{UF} is the volume of ultrafiltrate derived from the small pores, V_{Drain} is the volume of fluid drained from the peritoneal cavity, V_{Fill} is the fill volume (volume instilled into the peritoneal cavity), Cₚₗₛ^{Na} is the concentration of sodium in plasma, C_{Eff}^{Na} is the concentration of sodium in the incoming peritoneal dialysis fluid (PDF), and C_{Eff}^{Na} is the concentration of the sodium in the PD effluent.

The free water volume (providing a measure of aquaporin function) may then be identified by simply subtracting the small pore ultrafiltrate volume (V F) from the total UF volume (V_{Tot}^{UF}), as represented by:

$${V}_{FW}^{UF} = {V}_{Tot}^{UF} - {V}_{Sp}^{UF} \qquad Eq . 34$$

The need for sodium measurements may be avoided, by making the approximation that conductivity of PD fluid is determined in large part by its sodium content, therefore the proportionality constant between sodium concentration and conductivity cancels out. Thus, Eq. 33 may be rewritten as:

$${V}_{Sp}^{UF} = \frac{{V}_{Drain} \cdot {σ}_{Eff} - {V}_{Fill} \cdot {σ}_{PDF}}{{σ}_{pls}} \qquad Eq . 35$$

where σ_{Eff} is the conductivity of PD effluent, σ_{PDF} is the conductivity of fresh PD fluid, and σₚₗₛ is the conductivity of the plasma.

Furthermore, if the dwell phase is of sufficient duration (e.g., about 2 hours or longer) the concentration of sodium in the peritoneal cavity will be very close to the plasma concentration. Thus, the conductivity of the drained fluid of a current or previous PD cycle may be used as a substitute for measurement of plasma conductivity (i.e. σ_{drain}=σₚₗₛ). Therefore, Eq. 35 may be solved to get ultrafiltrate derived from the small pores V_{Sp}^{UF}, which may be subtracted from total UF volume (V_{Tot}^{UF}) to get the free water volume of the ultrafiltration (V_{FW}^{UF}).

Now referring to process 800 and FIG. 8, the method of determining free water volume of the ultrafiltration may begin with step 802, which can include measuring a conductivity of the PD effluent during a drain phase. For example, this conductivity measurement may be performed by PD-ECM 410. This conductivity measurement may be used for σ_{drain}=σₚₗₛ. Process 800, at step 804 can then include measuring a conductivity of fresh PD fluid during the fill phase. For example, this conductivity measurement may be performed by PD-ECM 410. This conductivity measurement may be used for σ_{PDF}. Process 800, at step 806 can include withdrawing one or more samples of PD effluent during the dwell phase and taking sensor measurements of the conductivity of the PD effluent sample(s), which conductivity measurement(s) may be for σ_{Eff}. Step 806 may be performed separately or as part of steps 602 and 604 of process 600. For example, sensor measurements of PD-ECM 410 may include both glucose and conductivity of the PD effluent samples. Process 800, at step 808 can include determining the free water volume of the ultrafiltration. For example, as discussed herein, with the conductivity measurements along with the known or measured volume measurements for the PD fill and PD drain, the free water volume of the ultrafiltration (VF) can be determined according to Eq. 35 and 34.

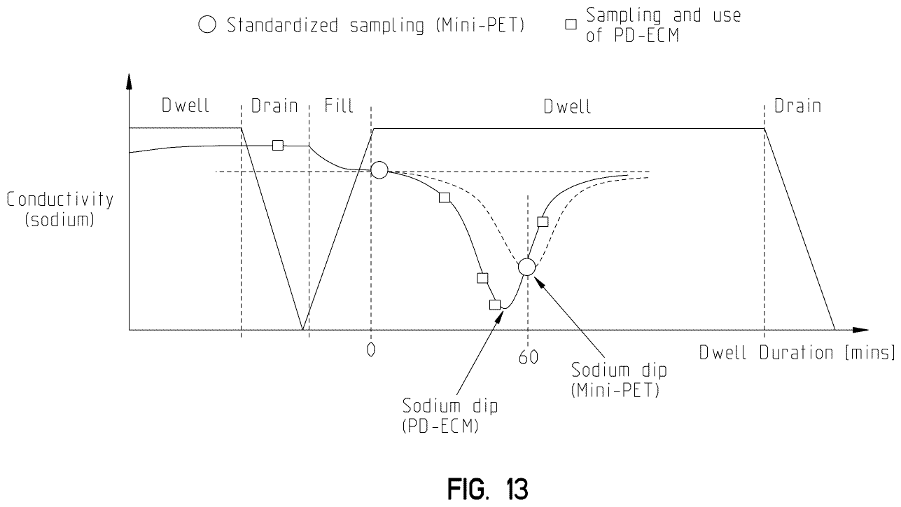

The difference between the conventional manual method of the mini-PET and the PD-ECM based method, described herein, is illustrated in the FIG. 13, which describes identification of the sodium dip by mini-PET methods and PD-ECM 410. The Mini-PET involves 3 samples, the timing of the PD effluent sample standardized at one hour. Via the PD-ECM 410, and using any of the aforementioned sampling methods multiple samples of PD effluent may be obtained at different times, either side of the 1 hour standard. This enables the magnitude and the timing of the sodium dip to be better identified in an individual patient. As the PD-ECM method is automated, the procedure may be repeated on several cycles with adjustments to the sample timing of PD effluent, further improving accuracy and confidence in the reliability of the dip phenomenon.

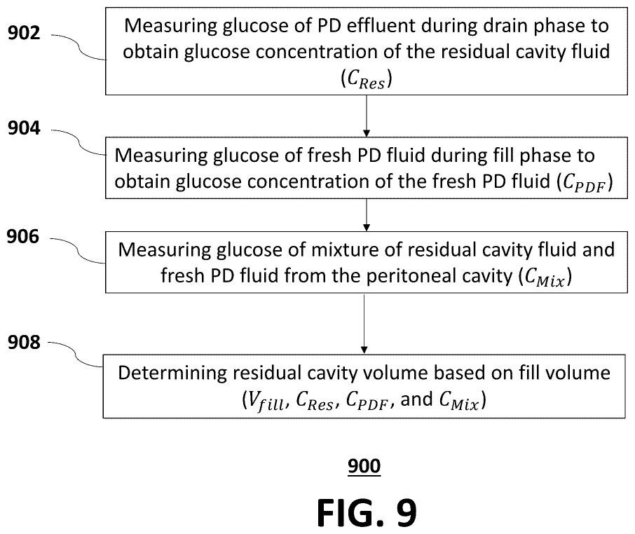

As described herein, in addition to enabling automated determination of membrane transport parameters, methods and system described herein can enable automated determination of a residual cavity volume. For instance, FIG. 9 is a flowchart of an exemplary process 900 for determining a residual cavity volume, according to one or more examples of the present disclosure. It will be recognized that any of the following steps may be performed in any suitable order, and that the process 900 may be performed in any suitable environment. Process 900 may be performed, for example, by PD system 100, controller 155, dialysis system 210, medical treatment system 400, or other devices and systems described herein. The descriptions, illustrations, and processes of FIG. 9 are merely exemplary and the process 900 may use other descriptions, illustrations, and processes for determining and using peritoneal membrane transport parameters.

Before getting into the details of the steps of process 900, a bit of additional information will be provided to assist in the explanation. Residual cavity volume arises due to incomplete drainage between consecutive PD cycles. One component of this volume (e.g., approx. 200 mL) is inaccessible even with optimal catheter placement. In any scenario, where the catheter is not optimally located in the cavity, the residual cavity volume following the drain phase may be considerably higher. This may be distinct from Tidal PD (TPD) therapy in which it may be desirable to not perform a full drain until the last drain phase.

When fresh PD fluid is introduced into the cavity containing residual volume, a simple mass balance equation may be representative as:

$${C}_{PDF} \cdot {V}_{Fill} + {C}_{Res} \cdot {V}_{Res} = {C}_{Mix} \cdot ( {V}_{Fill} + {V}_{Res} ) \qquad Eq . 36$$

where C_{PDF} is the glucose concentration of the fresh PD fluid, C_{Res} is the glucose concentration of the residual cavity fluid, C_{Mix} is the glucose concentration of a mixture of cavity fluid and fresh PD fluid, V_{Fill} is the fill volume (the volume of fresh PD fluid instilled in the cavity), and V_{Res} is the residual volume of the cavity to be determined.

Eq. 36 may be rearranged to solve for residual cavity volume, which yields:

$${V}_{Res} = \frac{{V}_{fill} ( {C}_{PDF} - {C}_{Mix} )}{{C}_{Mix} - {C}_{Res}} \qquad Eq . 37$$

Thus, according to Eq. 37, the residual cavity volume may be determined based on the V_{fill} and three glucose concentration measurements taken at appropriately selected times during a PD treatment cycle. The glucose concentration measurements may be performed by the PD-ECM. FIG. 13 is an illustration showing an example of timing of the glucose concentration measurements in relation to the different phases (drain, fill, and dwell). For example, as shown in FIG. 13, a glucose concentration for the residual cavity fluid (C_{Res}) may be obtained during (e.g., at the end) of a drain phase, a glucose concentration for the fresh PD fluid (C_{PDF}) may be obtained during the subsequent fill phase. The glucose concentration of a mixture of cavity fluid and fresh PD fluid (C_{Mix}) may be obtained in between the fill and dwell phase. As soon as fresh PD fluid (of high glucose concentration) enters the peritoneal cavity, glucose will begin to diffuse through small pores. To minimize this effect, in some implementations, the fill phase can be significantly shortened such that the cavity is partially filled to a volume of approximately 500 mL (e.g., 25% of a typical fill volume). After a short interval to allow mixing with the residual cavity fluid, a PD effluent may then be withdrawn to measure the glucose concentration for C_{Mix}.

In some implementations, a more rigorous approach to improve the accuracy of residual volume determination could be to take account of the absorption of glucose through the small pores while the fill is taking place. This has the advantage that the patient can be filled according to their normal fill volume and the mixing phase can be of an arbitrary duration.

Considering the flux of glucose in and out of the peritoneal cavity during the fill phase, then the glucose dynamics in the cavity may be approximated by the differential equation of Eq. 38 below. In this case, the effect of ultrafiltration is negligible compared with the flow rate of incoming PD fluid.

$$\frac{{dM}_{Mix}}{dt} = {C}_{Mix} \frac{{dV}_{PC}}{dt} + {V}_{PC} \frac{{dC}_{Mix}}{dt} = {C}_{PDF} \cdot Q - {C}_{Mix} \cdot {K}_{mtac} \qquad Eq . 38$$

where V_{PC} is the volume of the peritoneal cavity, Q is the flow rate of PD fluid into the cavity (negatively signed during the drain), K_{mtac} is the mass transfer area coefficient of glucose, all other variables are defined previously.

Since V_{PC}=V_{Res}+Q·t and

$$\frac{{dV}_{PC}}{dt} = Q ,$$

then Eq. 38 can be solved for C_{Mix} yielding:

$${C}_{Mix} = \frac{{C}_{PDF} \cdot Q}{{K}_{mtac} + Q} \cdot [ 1 - {( \frac{[ {V}_{Res} + Q \cdot t ]}{{V}_{Res}} )}^{γ} ] + {C}_{Res} \cdot {( \frac{[ {V}_{Res} + Q \cdot t ]}{{V}_{Res}} )}^{γ} \qquad Eq . 39$$

$$where$$

$$γ = \frac{- Q}{{K}_{mtac} + Q} \qquad Eq . 40$$

To illustrate the relationship with the simpler expression of Eq. 37, then by setting K_{mtac}=0 and V_{Fill}=Q·t, Eq. 39 reduces to:

$${C}_{Mix} = {C}_{PDF} ( 1 - \frac{{V}_{Res}}{{V}_{Res} + {V}_{Fill}} ) + {C}_{Res} \frac{{V}_{Res}}{{V}_{Res} + {V}_{Fill}} \qquad Eq . 41$$

Rearrangement of Eq. 41 for V_{Res} thus leads back to Eq. 37.

To apply Eq. 39 for the purposes of determining residual cavity volume, may require further treatment. For example, the mass transfer area coefficient for glucose, K_{mtac}, varies as the peritoneal cavity is filled, as dialysis fluid comes in in contact with an increasingly larger proportion of the peritoneal cavity surface area. K_{mtac} may be calculated by leveraging additional processes of the present disclosure, for example, determining of the glucose transport constant τ_{Glu}. Using the same approximation described previously, whereby the ratio of mass transfer area coefficient to cavity volume remains constant in an individual patient, yields:

$${K}_{mtac} = \frac{{V}_{Res} + Q \cdot t}{{τ}_{G l u}} \qquad Eq . 42$$

Eq. 42 may then be plugged into Eq. 39, and as Eq. 39 is transcendental, one or more suitable numerical methods may be applied to return the residual cavity volume(V_{Res}).

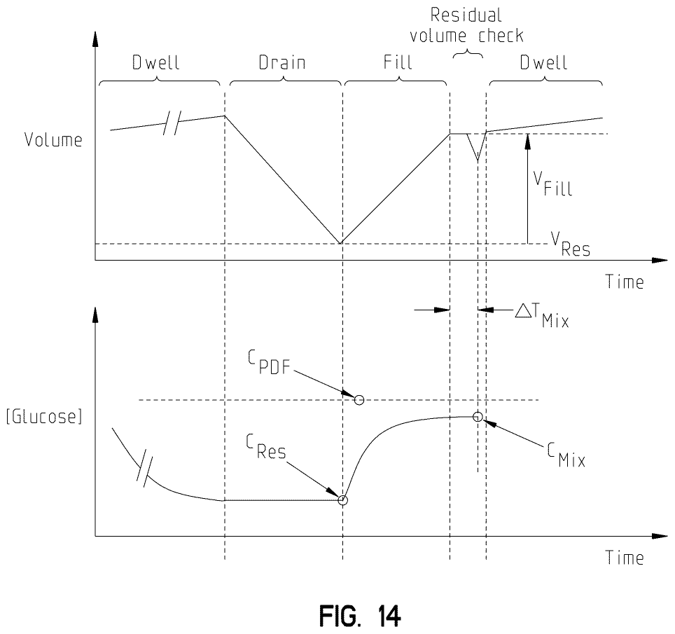

Now referring to process 900 and FIG. 9, the method of determining residual cavity volume may begin with step 902, which can include measuring glucose of PD effluent during the drain phase to obtained glucose concentration of the residual cavity fluid (C_{Res}). The timing of the measurement during the dwell phase may be at the end of the drain phase, as illustrated in FIG. 14. However, in some implementations the timing of this measurement during the dwell phase may be adjusted. Process 900, at step 904 can include measuring glucose of fresh PD fluid during fill phase to obtain glucose concentration of the fresh PD fluid (C_{PDF}). The timing of this measurement during the fill phase may be near the start of the fill phase, as illustrated in FIG. 14. However, in some implementations the timing of this measurement may be adjusted. Process 900, at step 906 can include measuring glucose of a mixture of residual cavity fluid and fresh PD fluid from the peritoneal cavity (C_{Mix}). As described herein, the timing of this measurement may vary. For example, in some implementations, the C_{Mix} glucose measurement may be taken at any point during the dwell phase after a normal fill, or in some implementations, the C_{Mix} glucose measurement may be taken a short time (to allow for mixing) after completing a shortened fill phase. Process 900, at step 908 can include determining residual cavity volume based on fill volume (V_{fill}, C_{Res}, C_{PDF}, and C_{Mix}). For example, for implementations that utilize a shortened fill phase, the residual cavity volume may be determined based on Eq. 37. For implementations that utilize a normal fill phase and the C_{Mix} glucose measurement is taken at any point during the dwell phase, the residual cavity volume may be determined based on Eq. 42 and Eq. 39 while additional utilizing the determined glucose transport constant τ_{Glu}.

As described herein, over time, typically in the space of months to years, peritoneal membrane transport characteristics of a PD patient change. These changes usually impact the membranes ability to transport solutes, water, and sodium. When membrane transport characteristics deteriorate significantly, leading to membrane failure, the patient may no longer be able to rely on PD treatment and may have to switch to an alternative form of dialysis (e.g., hemodialysis). There are several peritoneal membrane pathologies (e.g., encapsulating peritoneal sclerosis, fibrosis, and angiogenesis) and their identification is important for prognosis as well as treatment interventions to mitigate and slow membrane failure.

The relative magnitude of the membrane transport parameters and other determinable PD parameters, for example, blood glucose (C_{b}), creatine rate constant (τ_{Creat}), urea rate constant (τ_{Urea}), glucose transport constant (τ_{Glu}), the product of average reflection coefficient and UF coefficient ((σ_{Av}·k_{UF}), and rate of fluid absorption from the peritoneal cavity (J_{v_Abs}), free water volume of ultrafiltration (V_{FW}^{UF}), and residual cavity volume (RCV), which can be automatically determined according to the present disclosure, may be reflective of different membrane pathologies.

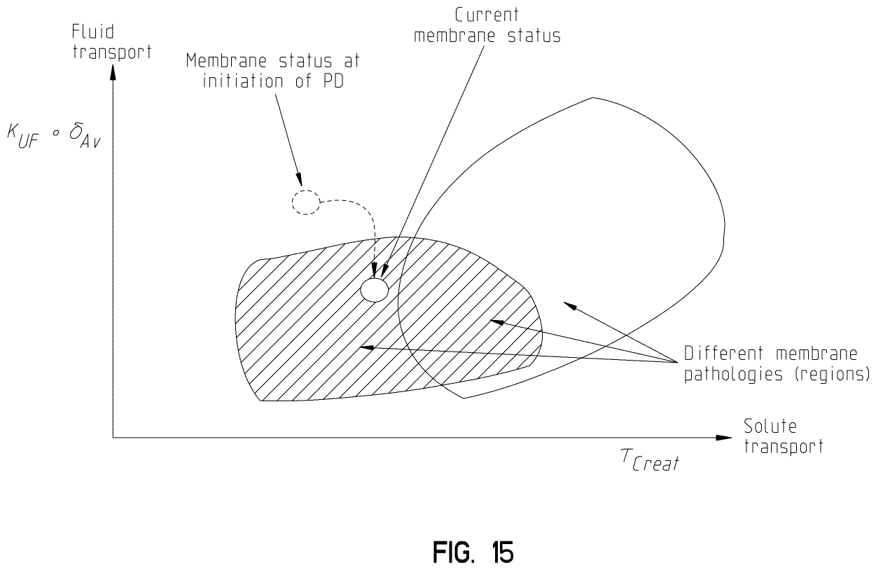

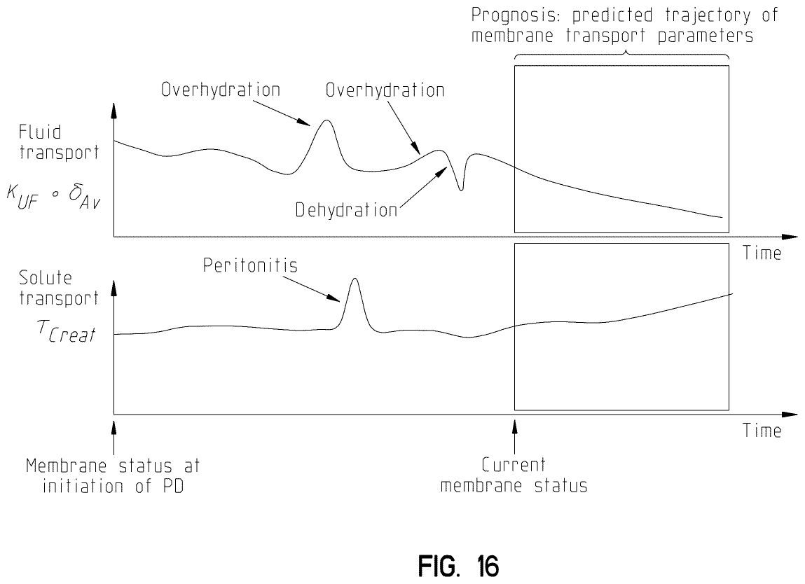

For example, FIG. 15 shows a hypothetical representation of membrane parameters (τ_{Creat}, σ_{Av}·k_{UF}), and some membrane pathologies. FIG. 15 illustrates an example of potential mapping of peritoneal membrane transport parameters to membrane pathologies. In some instances, the example in FIG. 15 could be extended to a 3-D region involving another membrane transport parameter. The ability to easily and routinely determine and track membrane transport parameters for PD patients, enabled by the methods and systems of the present disclosure, should enable mapping of membrane parameters to membrane pathologies along the lines of FIG. 15. Tracking on a daily basis can become practical as it introduces minimal, if any, extra burden or operating costs. Furthermore, the methods described herein, enable membrane transport parameter determination, without loss of treatment outcome quality as a consequence of the PD effluent sampling process. With high resolution tracking of membrane transport parameters, according to the present disclosure, identification of transient phenomena that are likely to influence the transport of solutes and water, e.g., a change in hydration status or an episode of peritonitis, may be possible. For example, FIG. 16 illustrates an example plot of membrane transport parameters (e.g., τ_{Creat}, σ_{Av}·k_{UF}) versus time. As illustrated in FIG. 16, increases or decreased in the fluid transport parameters (σ_{Av}·k_{UF}) may be used to identify a patient's hydration status (e.g., overhydration or dehydration). Additionally, a rapid increase in a solute transport parameter (e.g., τ_{Creat}) may be used to identify peritonitis, for example, as illustrated in FIG. 16. In this case, at the onset of such an unexpected spike, the control system may alert the patient and/or the healthcare professional.

Equipped with sufficient history of how membrane parameters are changing with time, and improved knowledge of their interrelationship, may enable a prediction of the future trajectories of membrane parameters (e.g., as illustrated in FIG. 16). This may in turn provide a useful prognostic indicator for many stakeholders. For example, this information could be used to trigger treatment intervention to help slow progression of membrane failure or facilitate advance planning of modality transition (e.g., transition to HD), when PD will no longer be possible.

In some implementations of the present disclosure, the control system may use one or more ML—AI algorithms/models (e.g., a neural network) to update the PD prescriptions. For example, the control system may provide as input the sensor measurements from the dwell phase and/or the original PD prescription into the one or more ML—AI algorithms, and the one or more ML—AI algorithms may provide an output indicating the updated PD prescription. For instance, the output may indicate to increase or decrease one or more of the glucose concentration, the dwell time, and/or the fill volume of the PD prescription.

In some variations, the control system may use the sensor measurements for one or more additional tasks. For instance, using one or more additional ML—AI algorithms/models (e.g., a neural network), the control system may determine a future time (e.g., in two weeks, two months, or even further into the future) that the peritoneal membrane (e.g., peritoneal lining 452) will fail. For example, as mentioned above, after continuous use of PD treatment, the peritoneal membrane of the patient may eventually fail. Using the one or more additional ML—AI algorithms and the sensor measurements, the control system may determine the future time that the peritoneal membrane will fail.

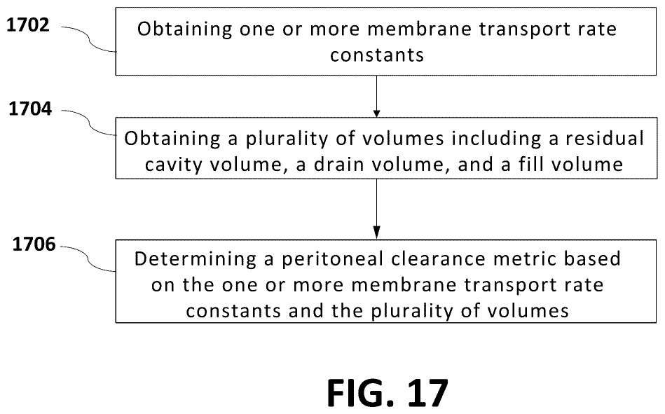

As described herein, in addition to enabling automated determination of membrane transport parameters, methods and system described herein can enable automated determination of a peritoneal clearance metric that may be used to assess the adequacy of the PD treatment for the patient. For instance, FIG. 17 is a flowchart of an exemplary process 1700 for determining a peritoneal clearance metric, according to one or more examples of the present disclosure. It will be recognized that any of the following steps may be performed in any suitable order, and that the process 1700 may be performed in any suitable environment. Process 1700 may be performed, for example, by PD system 100, controller 155, dialysis system 210, medical treatment system 400, or other devices and systems described herein. The descriptions, illustrations, and processes of FIG. 17 are merely exemplary and the process 1700 may use other descriptions, illustrations, and processes for determining and using peritoneal membrane transport parameters.

Before getting into the details of the steps of process 1700, a bit of additional information will be provided to assist in the explanation. Specifically, it may be desired to automatically track the adequacy and/or effectiveness of patients undergoing PD treatments, including the adequacy of the current PD dialysate prescription. For example, as mentioned above, over time, peritoneal membrane function may deteriorate, which may change the efficiency with which toxins are removed from the patient. The amount of toxins that are removed from the patient may be measured as a peritoneal clearance metric, and the adequacy or effectiveness of the PD treatment may be based on this peritoneal clearance metric. Therefore, using the peritoneal adequacy that may be based on the peritoneal clearance metric, a clinician may determine to adjust and/or change the individualized peritoneal dialysis prescription (e.g., perform step 608 of process 600) and/or change the treatment options for the patient altogether (e.g., move the patient onwards from PD treatment to another type of treatment such as HD treatment).

Traditionally, the peritoneal clearance metric may be manually determined by a clinician. For instance, a patient may go to a clinic a few times a year, and the clinician may measure the peritoneal clearance metric to determine the adequacy and effectiveness of the PD treatment. Automated procedures for determining the adequacy of a PD treatment avoid the need visit a clinic specifically. Additionally, as adequacy can be tracked on a more frequent basis with automated procedures, this facilitates advance planning of modality transition (e.g., transition to HD), when PD may no longer be possible for a patient.

To determine the peritoneal clearance metric, one or more of the processes and/or the peritoneal membrane transport parameters may be utilized. For instance, process 1700 may use the membrane transport rate constants τ and a plurality of volumes including the residual cavity volume that is determined using process 900 to determine the peritoneal clearance metric.

For example, assuming that R denotes the dialysate to plasma (D/P) ratio, the temporal variation of the D/P ratio may be expressed as:

$${R}_{〈 i 〉} ( t ) = {R}_{s s} + ( {R}_{0} - {R}_{s s} ) \cdot {e}^{t / {τ}_{〈 i 〉}} \qquad Eq . 43$$

where R₍ᵢ₎(t) is the temporal variation of the D/P ratio for the i^{th} solute, Rₛₛ is the D/P in steady state (e.g., when plasma and dialysate concentrations of a solute are equal, which implies that Rₛₛ is 1), R₀ is the initial D/P, and τ₍ᵢ₎ are the membrane transport rate constants for the i^{th} solute. For instance, τ₍ᵢ₎ may be and/or include the creatine rate constant (τ_{Creat}), the urea rate constant (τ_{Urea}), the glucose transport constant (τ_{Glu}), and/or other rate constants described above. In some embodiments, τ₍ᵢ₎ may indicate the urea and/or creatinine rate constants. As mentioned above, in some examples, the urea and creatinine rate constants may be obtained (e.g., derived) from the glucose transport constant (e.g., from Eqs. 12 and 13 above). The glucose transport constant may be obtained via the PD-ECM, which is also described above.

For example, the membrane transport rate constants for a given solute such as creatinine or urea, are determined indirectly via the glucose rate constant by leveraging the relationships of Eqs. 12-13 above. Eq. 43 has similarity to Eqs. 10-11, but is modified to take into account the residual volume in the cavity. The residual volume impacts R₀ (the initial D/P ratio) according to the below expression:

$${R}_{0} = \frac{{V}_{Res}}{{V}_{Res} + {V}_{Fill}} \qquad Eq . 44$$

where V_{Res} is the residual cavity volume and V_{Fill} is the fill volume.

Therefore, at the end of the dwell phase (e.g., immediately prior to the drain phase), the time t would equal the dwell time T_{Dwell} and therefore, the final D/P ratio may be calculated based on Eq. 43. Following, the peritoneal clearance metric by definition is:

$${K}_{〈 i 〉} = {R}_{〈 i 〉} ( {T}_{D w e l l} ) \cdot {V}_{D r a i n} \qquad Eq . 45$$

where K₍ᵢ₎ is the clearance (e.g., the peritoneal clearance metric) of the i^{th} solute and V_{Drain} is the volume of effluent drained from the cavity.

Taking the above Eq. 43-45, the peritoneal clearance metric K₍ᵢ₎ may be determined by:

$${K}_{〈 i 〉} = [ 1 + ( \frac{{V}_{Res}}{{V}_{Res} + {V}_{Fill}} - 1 ) \cdot {e}^{{T}_{D w e l l} / {τ}_{〈 i 〉}} ] \cdot {V}_{Drain} \qquad Eq . 46$$

In view of the above and now referring to process 1700 and FIG. 17, the method of determining the peritoneal clearance metric may begin with 1702, which can include obtaining one or more membrane transport rate constants τ₍ᵢ₎. For instance, as described in FIG. 7 and process 700, the membrane transport rate constants such as the creatine rate constant (τ_{Creat}), the urea rate constant (τ_{Urea}), and/or the glucose transport constant (τ_{Glu}) may be determined. Process 1700, at step 1704 can include obtaining a plurality of volumes including a residual cavity volume, a drain volume, and a fill volume. For example, based on using FIG. 9 and process 900, the residual cavity volume V_{Res} may be obtained using Eq. 37. The drain volume V_{Drain} and the fill volume V_{Fill} may also be obtained, which is described above. Following, process 1700, at step 1706 can determine a peritoneal clearance metric based on the one or more membrane transport rate constants and the plurality of volumes. For example, using Eq. 43-46, the peritoneal clearance metric may be determined. Then, returning back to FIG. 6 and process 600, based on determining the peritoneal clearance metric, an individualized peritoneal dialysis prescription for the patient may be updated. Additionally, and/or alternatively, as mentioned above, further actions may be taken such as changing the treatment options for the patient (e.g., move the patient onwards from PD treatment to another type of treatment such as HD treatment).

It will be appreciated that the various machine-implemented operations described herein may occur via the execution, by one or more respective processors, of processor-executable instructions stored on a tangible, non-transitory computer-readable medium, such as a random access memory (RAM), read-only memory (ROM), programmable read-only memory (PROM), and/or another electronic memory mechanism. Thus, for example, operations performed by any device described herein may be carried out according to instructions stored on and/or applications installed on the device, and via software and/or hardware of the device.

All references, including publications, patent applications, and patents, cited herein are hereby incorporated by reference to the same extent as if each reference were individually and specifically indicated to be incorporated by reference and were set forth in its entirety herein.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present application covers further embodiments with any combination of features from different embodiments described above and below.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

1. A method of updating a peritoneal dialysis (PD) prescription of a patient, comprising:
withdrawing a plurality of samples of peritoneal dialysis effluent from the patient's peritoneal cavity periodically during one or more dwell phases of one or more peritoneal dialysis treatment cycles;
obtaining sensor measurements of the samples of peritoneal dialysis effluent;
determining one or more peritoneal membrane transport parameters based on the sensor measurements; and
updating an individualized peritoneal dialysis prescription for the patient, to be used for a later peritoneal dialysis treatment, based on the one or more peritoneal membrane transport parameters.

2. The method of claim 1, further comprising:
performing the later peritoneal dialysis treatment for the patient using the updated individualized peritoneal dialysis prescription.

3. The method of claim 1, wherein withdrawing the plurality of samples comprises withdrawing the plurality of samples during a plurality of different dwell phases of the peritoneal dialysis treatment, and
wherein at least one of a dwell duration and a glucose concentration is different for the plurality of different dwell phases.

4. The method of claim 1, wherein the sensor measurements of the samples of the peritoneal dialysis effluent indicate one or more glucose concentrations and one or more conductivity levels of one or more electrolyte concentrations of the samples of the peritoneal dialysis effluent.

5. The method of claim 4, wherein the one or more peritoneal membrane transport parameters comprise a glucose transport constant, and wherein determining the glucose transport constant comprises:
performing an optimization procedure that fits a volume model for a peritoneal cavity fluid volume to the glucose concentrations from the sensor measurements of the samples of peritoneal dialysis effluent.

6. The method of claim 5, wherein the volume model is either a fixed volume model represented by the following equation:
$${R}_{G l u}^{D / D 0} ( t ) = \frac{{C}_{d}}{{C}_{d} ( 0 )} = \frac{{C}_{b}}{{C}_{d} ( 0 )} - ( \frac{{C}_{b}}{{C}_{d} ( 0 )} - \frac{{C}_{d} ( 0 )}{{C}_{d} ( 0 )} ) {e}^{- \frac{t}{{τ}_{G l u}}} = \frac{{C}_{b}}{{C}_{d} ( 0 )} + ( 1 - \frac{{C}_{b}}{{C}_{d} ( 0 )} ) {e}^{- \frac{t}{{τ}_{G l u}}} ;$$
or the volume model is a variable volume represented by the following equation:
$${R}_{G l u}^{D / D 0} ( t ) = \frac{{C}_{b}}{{C}_{d} ( 0 )} + \frac{{V}_{Tot}^{PC} ( 0 )}{{V}_{Tot}^{PC} ( t )} ( 1 - \frac{{C}_{b}}{{C}_{d} ( 0 )} ) {e}^{- \frac{t}{{τ}_{glu}} ;}$$
wherein R_{Glu}^{D/D0}(t) is the temporal variation of glucose in PD effluent during the dwell phase, C_{b} is the concentration of glucose in blood, C_{d} is the concentration of glucose in the PD fluid contained in the peritoneal cavity, τ_{Glu} is the glucose transport constant, and V_{Tot}^{PC} is the total volume of fluid in the peritoneal cavity.

7. The method of claim 5, wherein the one or more peritoneal membrane transport parameters comprise a creatine rate constant (τ_{Creat}) and a urea rate constant (τ_{Urea}), which are determined based on the glucose transport constant and universal constants relating the glucose rate constant to the creatine and urea rate constant.

8. The method of claim 4, wherein the one or more peritoneal membrane transport parameters comprise a rate of fluid absorption from the peritoneal cavity (J_{v_Abs}) and product of an average reflection coefficient (σ_{Av}) and an ultrafiltration (UF) coefficient (k_{UF}), and wherein determining J_{v_Abs}, and the product of σ_{Av} and k_{UF}, comprises:
determining a glucose transport constant by performing an optimization procedure that fits a volume model for peritoneal cavity fluid volume to the glucose concentrations from the sensor measurements of the samples of peritoneal dialysis effluent;
obtaining UF volumes for the dwell phases where the sensor measurements of the samples of PD effluent are obtained; and
determining J_{v_Abs} and a product of σ_{Av} and k_{UF} based on the glucose transport constant, the UF volumes, and the glucose concentrations from the sensor measurements.

9. The method of claim 8, wherein J_{v_Abs} and the product of σ_{Av} and k_{UF} are determined utilizing the following equation:
$${V}_{Tot}^{P C} ( t ) = {V}_{fill} + {τ}_{g l u} \cdot {k}_{UF} \cdot {σ}_{A v} \cdot Δ {P}_{Osm} ( 0 ) \cdot ( 1 - {e}^{- \frac{t}{{τ}_{g l u}}} ) - {J}_{v \_ Abs} \cdot t ;$$
wherein V_{Tot}^{PC} is a total volume of fluid in the peritoneal cavity, V_{fill} is a volume of PD fluid supplied during the fill phase, ΔPₒₛₘ(0) is an initial transperitoneal differential osmotic pressure, and t is time.

10. The method of claim 1, wherein withdrawing the plurality of samples of the peritoneal dialysis effluent from the patient's peritoneal cavity comprises:
providing, by a computing device and to a pump of a peritoneal dialysis system, first instructions to withdraw the plurality of samples; and
subsequent to providing the first instructions, providing, by the computing device, second instructions to fill the patient's peritoneal cavity with fresh peritoneal dialysis effluent, and
wherein obtaining the sensor measurements comprises obtaining the sensor measurements by the computing device and in response to providing the first instructions to withdraw the plurality of samples.

11. The method of claim 1, wherein the peritoneal dialysis system comprises one or more buffer zones in fluid communication with the pump, and wherein withdrawing the plurality of samples of the peritoneal dialysis effluent from the patient's peritoneal cavity comprises:
providing, by a computing device and to a pump of a peritoneal dialysis system, first instructions to withdraw the plurality of samples; and
subsequent to providing the first instructions, providing, by the computing device, second instructions to fill the patient's peritoneal cavity with effluent from the one or more buffer zones, and
wherein obtaining the sensor measurements comprises obtaining the sensor measurements by the computing device and in response to providing the first instructions to withdraw the plurality of samples.

12. The method of claim 1, wherein the individualized peritoneal dialysis prescription indicates a PD fluid glucose concentration, a PD fluid volume, and a dwell time for the patient, and wherein updating the individualized peritoneal dialysis prescription for the patient comprises adjusting at least one of the PD fluid glucose concentration, the PD fluid volume, or the dwell time based on the one or more peritoneal membrane transport parameters.

13. The method of claim 12, further comprising:
adjusting the at least one of the PD fluid glucose concentration, the PD fluid volume, or the dwell time based on using the one or more peritoneal membrane transport parameters and one or more peritoneal dialysis prescription machine learning (ML)—artificial intelligence (AI) algorithms to determine an adjustment to the PD fluid glucose concentration, the PD fluid volume, or the dwell time; and
using the one or more peritoneal membrane transport parameters and one or more peritoneal dialysis failure ML—AI algorithms to determine a predicted time period for a membrane of the patient's peritoneal cavity to fail.

14. A peritoneal dialysis (PD) system, comprising:
a pump configured to be fluidly connected to a PD patient line, wherein the pump is configured to withdraw samples of PD effluent periodically during a dwell phase of a peritoneal treatment for a patient;
a PD sensor in fluid communication with the patient line and configured to take sensor measurements of PD effluent entering or exiting the PD patient line;
a computing device, programmed to perform a peritoneal dialysis treatment using an individualized peritoneal dialysis prescription, wherein the individualized peritoneal dialysis prescription indicates a PD fluid glucose concentration, a PD fluid volume, and a dwell time for the patient;
wherein the computing device is configured to:
calculate one or more peritoneal membrane transport parameters based on a plurality of sensor measurements;
update the individualized peritoneal dialysis prescription for the patient based on the one or more peritoneal membrane transport parameters; and
perform a subsequent peritoneal dialysis treatment for the patient using the updated individualized peritoneal dialysis prescription.

15. A method of determining a residual cavity volume of a peritoneal dialysis (PD) patient, comprising:
measuring glucose of a PD effluent during a drain phase to obtained glucose concentration of a residual cavity fluid (C_{Res});
measuring glucose of a fresh PD fluid during a fill phase to obtain glucose concentration of the fresh PD fluid (C_{PDF});
measuring glucose of a mixture of the residual cavity fluid and the fresh PD fluid from the peritoneal cavity (C_{Mix}); and
determining the residual cavity volume based on a fill volume (V_{fill}), C_{Res}, C_{PDF}, and C_{Mix}.

16. The method of claim 15, wherein determining the residual cavity volume (V_{Res}) utilizes the following equation:
$${V}_{Res} = \frac{{V}_{fill} ( {C}_{PDF} - {C}_{Mix} )}{{C}_{Mix} - {C}_{Res}} .$$

17. The method of claim 15, further comprising:
determining one or more membrane transport rate constants; and
determining a peritoneal clearance metric based on the one or more membrane transport rate constants and the determined residual cavity volume.

18. A method of determining a peritoneal clearance metric indicating an adequacy of a of a peritoneal dialysis (PD) treatment for a patient, comprising:
obtaining one or more membrane transport constants τ₍ᵢ₎;
obtaining a plurality of volumes associated with the PD treatment, wherein the plurality of volumes comprises a residual cavity volume V_{Res}, a drain volume V_{Drain}, and a fill volume V_{fill}; and
determining a peritoneal clearance metric based on the one or more membrane transport constants, the drain volume, and the fill volume.

19. The method of claim 18, wherein the one or more membrane transport constants τ₍ᵢ₎ comprises a creatine rate constant (τ_{Creat}), a urea rate constant (τ_{Urea}), and/or a glucose transport constant (τ_{Glu}).

20. The method of claim 18, wherein determining the peritoneal clearance metric utilizes the following equation:
$${K}_{〈 i 〉} = 1 + ( \frac{{V}_{Res}}{{V}_{Res} + {V}_{Fill}} - 1 ) \cdot {e}^{{T}_{Dwell} / {τ}_{〈 i 〉}} \cdot {V}_{Drain}$$
where K₍ᵢ₎ is the peritoneal clearance metric and T_{Dwell} is a dwell time associated with the PD treatment.

21. The method of claim 18, further comprising:
updating an individual peritoneal dialysis prescription for the patient based on the determined peritoneal clearance metric; and
using the updated individual peritoneal dialysis prescription for a subsequent PD treatment for the patient.
